(19) United States
(12) Patent Application Publication
(10) Pub. No.: US 2010/0154082 A1
(43) Pub. Date: Jun. 17, 2010

(54) SHADE TOLERANCE IN PLANTS

(75) Inventors: Shing Kwok, Woodland Hills, CA (US); Amy Jo Miyamoto, Belleville, WI (US)

Correspondence Address: FISH & RICHARDSON P.C., P.O. BOX 1022, MINNEAPOLIS, MN, 55440-1022, US

(73) Assignee: Ceres, Inc., Newbury Park, CA (US)

(21) Appl. No.: 12/300,284

(22) PCT Filed: May 10, 2007
(86) PCT No.: PCT/US07/68705

§ 371 Date: May 19, 2009

Related U.S. Application Data
(60) Provisional application No. 60/799,404, filed on May 10, 2006.

(51) Int. Cl. A01H 5/08; A01H 5/00; A01H 5/10; C12N 5/10; A01G 1/00

(52) U.S. Cl. 800/306; 800/298; 800/314; 800/320; 800/320.2; 800/320.1; 435/419; 47/58.1 R

(57) ABSTRACT

Materials and methods for increasing shade tolerance in plants are disclosed. For example, nucleic acids encoding shade-tolerance polypeptides are disclosed as well as methods for using such nucleic acids to transform plant cells. Also disclosed are plants having increased shade tolerance and plant products produced from plants having increased shade tolerance.

### TECHNICAL FIELD

This invention relates to materials and methods involved in shade tolerance in plants. For example, this document provides plants having increased shade tolerance as well as materials and methods for making plants having increased shade tolerance and plant products derived from plants having increased shade tolerance.

### INCORPORATION-BY-REFERENCE & TEXT

The material on the accompanying electronic file is hereby incorporated by reference into this application. The accompanying file is titled, 18207017WO1Sequencelisting.txt, was created on May 10, 2007. The file named 18207017WO1Sequencelisting.txt is 132 KB. The file can be accessed using Microsoft Word on a computer that uses Windows OS.

### BACKGROUND

Higher plants depend on the acquisition of light energy for their survival. Since plants cannot choose their surroundings, they are forced to adapt their growth to ambient light conditions and have evolved complex mechanisms for monitoring the quantity and quality of the surrounding light. For example, many kinds of plants respond to growth under dense canopies or at high densities by manifesting a “shade-avoidance response”, i.e., by growing faster and taller (Cerdan and Chory, 2003). Densely planted crops tend to place energy into stem and petiole elongation to lift the leaves into the sunlight rather than putting energy into storage or reproductive structures. The shade-avoidance response negatively affects crop yields by reducing the amount of harvestable products such as seeds, fruits and tubers. In addition, tall spindly plants tend to be less wind resistant and lodge more easily, further reducing crop yield.

There is a continuing need for plants that can thrive under less than optimal environmental conditions. One strategy to improve a plant's ability to withstand suboptimal environmental conditions relies upon traditional plant breeding methods. Another approach involves genetic manipulation of plant characteristics through the introduction of exogenous nucleic acids conferring a desirable trait.

### SUMMARY

The spectral energy distribution of daylight is dramatically altered by vegetation. Light reflected from neighboring vegetation is depleted in red (R) wavelengths, but remains rich in far-red (FR) wavelengths. A useful parameter to describe the natural light environment is the ratio of light in the red (R) wavelengths to the light in the far-red (FR) wavelengths (R:FR ratio). The R:FR ratio of daylight is typically about 1.15; the R:FR ratios reported underneath canopies of vegetation range from about 0.05 to about 0.7. Thus, the light in shady environments is enriched in FR wavelengths relative to the light in non-shady environments.

The invention features methods and materials related to increasing tolerance to far-red enriched (FRE) conditions in plants. FRE conditions can include growth conditions in which the R:FR ratio is less than 1.0 and that typically result in shade avoidance responses in the plants grown under those conditions. Under FRE-conditions, FRE-tolerant plants display a reduction in the level of shade avoidance responses relative to the level of shade avoidance responses in non-FRE-tolerant plants. The methods provided herein can include transforming a plant cell with a nucleic acid encoding an FRE-tolerance polypeptide, wherein expression of the polypeptide results in an increased level of FRE-tolerance. Plant cells produced using such methods can be grown to produce plants having increased FRE tolerance. Increasing the FRE-tolerance of plants can increase the crop yields of such plants, which may benefit both food consumers and producers.

Accordingly, plants having increased FRE-tolerance are provided. In one embodiment, a plant having increased FRE-tolerance can include a plant including an exogenous nucleic acid, the exogenous nucleic acid including a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, where the plant has a statistically significant difference in a response to far-red-enriched (FRE) light conditions as compared to the corresponding response in a control plant that does not include the regulatory region operably linked to the nucleotide sequence. The sequence identity can be 80%, 85%, 90%, 95% percent or greater.

In another embodiment, a plant having increased FRE-tolerance can include a plant comprising (a) a first exogenous nucleic acid including a nucleotide sequence encoding a polypeptide having 80 percent or greater sequence identity to an amino acid sequence selected from the group consisting of SEQ ID NO: 41 operably linked to a regulatory region and (b) a second exogenous nucleic acid including a nucleotide sequence encoding a polypeptide having 80 percent or greater sequence identity to an amino acid sequence selected from the group consisting of SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 operably linked to a regulatory region; where the plant has a statistically significant difference in a response to far-red-enriched (FRE) light conditions as compared to the corresponding response in a control plant that does not comprise the first and second exogenous nucleic acids. The sequence identity can be 80%, 85%, 90%, 95% percent or greater.

In one embodiment, a plant having increased FRE-tolerance can include a plant including an exogenous nucleic acid, the exogenous nucleic acid including a regulatory region operably linked to a nucleotide sequence encoding a including an amino acid sequence corresponding to SEQ ID NO: 41. In another embodiment, a plant having increased FRE-tolerance can include a plant including an exogenous nucleic acid, the exogenous nucleic acid including a regulatory region operably linked to a nucleotide sequence encoding a polypeptide including an amino acid sequence corresponding to SEQ ID NO: 43. In another embodiment, a plant having increased FRE-tolerance can include a plant including an exogenous nucleic acid, the exogenous nucleic acid including a regulatory region operably linked to a nucleotide sequence encoding a polypeptide wherein the Hidden Markov Model bit score of the amino acid sequence of said polypeptide is greater than 395, the HMM based on the amino acid sequences depicted in FIG. 1. The plant has a statistically significant difference in a response to far-red-enriched light conditions compared to a control plant that lacks the exogenous nucleic acid.

Also featured are progeny of any of the plants described above wherein the progeny has a statistically significant difference in a response to far-red-enriched (FRE) light conditions as compared to the corresponding response in a control plant that does not comprise said regulatory region operably linked to a nucleotide sequence.

Recombinant vectors are also provided. Recombinant vectors can include a described nucleic acid operably linked to a regulatory region. The regulatory region can be a promoter. The promoter can be a tissue-preferential, broadly expressing, or inducible promoter.

The plant can be a dicot. The plant can be a member of one of the following genera: Brassica spp., Brassica napus, Brassica rapa, Brassica oleracea, Glycine max, Gossypium spp., Gossypium hirsutum, Gossypium herbaceum, Helianthus annuus, Lactuca sativa, Medicago sativa.

The plant can be a monocot. The plant can be a member of one of the following genera: Avena sativa, Hordeum vulgare, Oryza sativa, Panicum virgatum, Secale cereale, Triticum aestivum, and Zea mays.

The statistically significant difference in a response to far-red-enriched (FRE) light conditions can be a difference in petiole length. In another aspect, the statistically significant difference in a response to far-red-enriched (FRE) light conditions can be a difference in hypocotyl length.

FRE conditions include a red:far-red (R:FR) ratio of less than 1.0, e.g., from about 0.05 to about 0.9, from about 0.10 to about 0.7, from about 0.10 to about 0.5, from about 0.10 to about 0.3. In one embodiment, the R:FR ratio can be 0.22. In another embodiment, the R:FR ratio can be 0.14.

The far-red-enriched (FRE) light conditions comprise can include continuous FRE conditions. In another aspect, far-red-enriched light conditions can include a pulse of FRE conditions, e.g., a pulse of FRE conditions that includes about 0.1 to about 8.0 hours of FRE conditions per day, a pulse of FRE conditions that includes about 0.2 to about 6.0 hours of FRE conditions per day, a pulse of FRE conditions that includes about 0.3 to about 3.0 hours of FRE conditions per day, a pulse of FRE conditions that includes about 0.4 to about 2.0 hours of FRE conditions per day, or a pulse of FRE conditions that includes 0.5 hours of FRE conditions per day. The 0.5 hour pulse of FRE conditions can include an 0.5 hour pulse of FRE conditions at the end of the day.

In another embodiment, a transgenic plant having increased FRE-tolerance can include a plant including an exogenous nucleic acid, the exogenous nucleic acid comprising a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, where the transgenic plant has a statistically significant difference in a response to far-red-enriched (FRE) light conditions as compared to the corresponding response in a control plant that does not include the regulatory region operably linked to the nucleotide sequence. The sequence identity is 80%, 85%, 90%, 95% percent or greater.

In one embodiment, a transgenic plant having increased FRE-tolerance can include a plant including an exogenous nucleic acid, the exogenous nucleic acid including a regulatory region operably linked to a nucleotide sequence encoding a including an amino acid sequence corresponding to SEQ ID NO: 41. In another embodiment, a transgenic plant having increased FRE-tolerance can include a plant including an exogenous nucleic acid, the exogenous nucleic acid including a regulatory region operably linked to a nucleotide sequence encoding a polypeptide including an amino acid sequence corresponding to SEQ ID NO: 43. In another embodiment, a transgenic plant having increased FRE-tolerance can include a plant including an exogenous nucleic acid, the exogenous nucleic acid including a regulatory region operably linked to a nucleotide sequence encoding a polypeptide wherein the Hidden Markov Model bit score of the amino acid sequence of said polypeptide is greater than 395, the HMM based on the amino acid sequences depicted in FIG. 1. The plant has a statistically significant difference in a response to far-red-enriched light conditions compared to a control plant that lacks the exogenous nucleic acid.

A method of producing a crop is also provided. The method includes: growing a plurality of plants including an exogenous nucleic acid comprising a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, where the plant has a statistically significant difference in a response to FRE light conditions as compared to the corresponding response in a control plant that does not comprise the regulatory region operably linked to a nucleotide sequence; and harvesting the crop from the plants.

In another embodiment, seeds, vegetative tissue, and fruit from transgenic plants having increased FRE-tolerance are provided. Seeds, vegetative tissue, and fruit can be from a transgenic plant including an exogenous nucleic acid, the exogenous nucleic acid including a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, where the transgenic plant has a statistically significant difference in a response to FRE light conditions as compared to the corresponding response in a control plant that does not include the regulatory region operably linked to the nucleotide sequence. The sequence identity can be 80%, 85%, 90%, 95% percent or greater.

In another embodiment, seeds, vegetative tissue, and fruit can be from a transgenic plant including (a) a first exogenous nucleic acid including a nucleotide sequence encoding a polypeptide having 80 percent or greater sequence identity to an amino acid sequence selected from the group consisting of SEQ ID NO: 41 and (b) a second exogenous nucleic acid including a nucleotide sequence encoding a polypeptide having 80 percent or greater sequence identity to an amino acid sequence selected from the group consisting of SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58; where the transgenic plant has a statistically significant difference in a response to far-red-enriched (FRE) light conditions as compared to the corresponding response in a control plant that does not comprise the regulatory region operably linked to the nucleotide sequence. The sequence identity is 80%, 85%, 90%, 95% percent or greater.

In some embodiments, seeds, vegetative tissue, and fruit can be from a transgenic plant including an exogenous nucleic acid, the exogenous nucleic acid including a regulatory region operably linked to a nucleotide sequence encoding a polypeptide wherein the Hidden Markov Model bit score of the amino acid sequence of said polypeptide is greater than 395, the HMM based on the amino acid sequences depicted in FIG. 1. The plant has a statistically significant difference in a response to red-enriched light conditions compared to a control plant that lacks the exogenous nucleic acid.

Also provided are articles of manufacture. An article of manufacture can include packaging material and seeds within the packaging material, the seeds including an exogenous nucleic acid, the exogenous nucleic acid including a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, where plants grown from the seeds have a statistically significant difference in a response to FRE light conditions as compared to the corresponding response in a control plant that does not comprise the regulatory region operably linked to said nucleotide sequence. In another aspect, the seeds included in the article of manufacture can include a regulatory region including a first transcription activator recognition site and a first promoter, the seeds further including: a second exogenous nucleic acid, the second exogenous nucleic including a second transcription activator recognition site and a second promoter, the second recognition site and the second promoter operably linked to a sequence causing seed infertility; and at least one activator nucleic acid encoding at least one transcription activator that binds to at least one of the recognition sites, each of the at least one transcription activator having a promoter operably linked thereto, where plants grown from the seeds are infertile.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an alignment of the amino acid sequence of Ceres Clone 37493 (SEQ ID NO: 43) with homologous and/or orthologous amino acid sequences gi|50929439 (SEQ ID NO: 46), Ceres gDNA 1494370 (SEQ ID NO: 45), gi|59611829 (SEQ ID NO: 58), gi|87241303 (SEQ ID NO: 56), gi|37904506 (SEQ ID NO: 48), gi|77745528 (SEQ ID NO: 52), gi|6651395 (SEQ ID NO: 50), gi|55442027 (SEQ ID NO: 51), gi|9789277 (SEQ ID NO: 55), gi|18461100 (SEQ ID NO: 47), gi|6002712 (SEQ ID NO: 57), gi|58201418 (SEQ ID NO: 49), gi|28629495 (SEQ ID NO: 53), gi|58201458 (SEQ ID NO: 54). Like reference symbols in the drawing indicates like elements.

### DETAILED DESCRIPTION

The spectral energy distribution of daylight is dramatically altered by vegetation. Light reflected from neighboring vegetation is depleted in red (R) wavelengths, but remains rich in far-red (FR) wavelengths. A useful parameter to describe the natural light environment is the ratio of light in the red (R) wavelengths to the light in the far-red (FR) wavelengths (R:FR ratio). The R:FR ratio of daylight is typically about 1.15; the R:FR ratios reported underneath canopies of vegetation range from about 0.05 to about 0.7. Thus, the light in shady environments is enriched in FR wavelengths relative to the light in non-shady environments.

The invention features methods and materials related to increasing tolerance to far-red enriched (FRE) conditions in plants. FRE conditions can include growth conditions in which the R:FR ratio is less than 1.0 and that typically result in shade avoidance responses in the plants grown under those conditions. Under FRE-conditions, FRE-tolerant plants display a reduction in the level of shade avoidance responses relative to the level of shade avoidance responses in non-FRE-tolerant plants.

The methods provided herein can include transforming a plant cell with a nucleic acid encoding an FRE-tolerance polypeptide, wherein expression of the polypeptide results in an increased level of FRE tolerance. Plant cells produced using such methods can be grown to produce plants having increased FRE tolerance.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include FRE-tolerance polypeptides. FRE-tolerance polypeptides can be effective to increase FRE-tolerance when expressed in a plant or plant cell. An increase in FRE-tolerance can be an increase in the level of FRE-tolerance relative to the corresponding level in a control plant. An FRE-tolerance polypeptide can be a transcription factor polypeptide, such as a putative zinc finger transcription factor protein. An FRE-tolerance polypeptide can be an enzyme, such as an S-adenosyl-L-methionine (SAM) dependent-salicylic acid carboxyl methyl transferase-like protein.

An FRE-tolerance polypeptide can comprise the amino acid sequence of Ceres Clone 258241 as set forth in SEQ ID NO: 41. Ceres Clone 258241 (SEQ ID NO: 41) is predicted to encode a putative zinc finger transcription factor. Transcription factors are a diverse class of proteins that regulate gene expression through specific DNA binding events. Transcription factors are involved in a variety of regulatory networks of genes in plants, including those genes responsible for perception of quality and quantity of light. Transcription factors include a number of characteristic structural motifs that mediate interactions with nucleic acids. Zinc finger and B-box motifs, which typically include one or more cysteine and histidine residues that can bind a zinc atom, can serve as structural platforms for DNA binding and/or protein-protein interactions.

Thus, an FRE-tolerance polypeptide can be an Arabidopsis polypeptide having the amino acid sequence set forth in SEQ ID NO: 41. Alternatively, an FRE-tolerance polypeptide can be a homolog, ortholog, or variant of the polypeptide having the amino acid sequence set forth in SEQ ID NO: 41. For example, an FRE-tolerance polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO: 41.

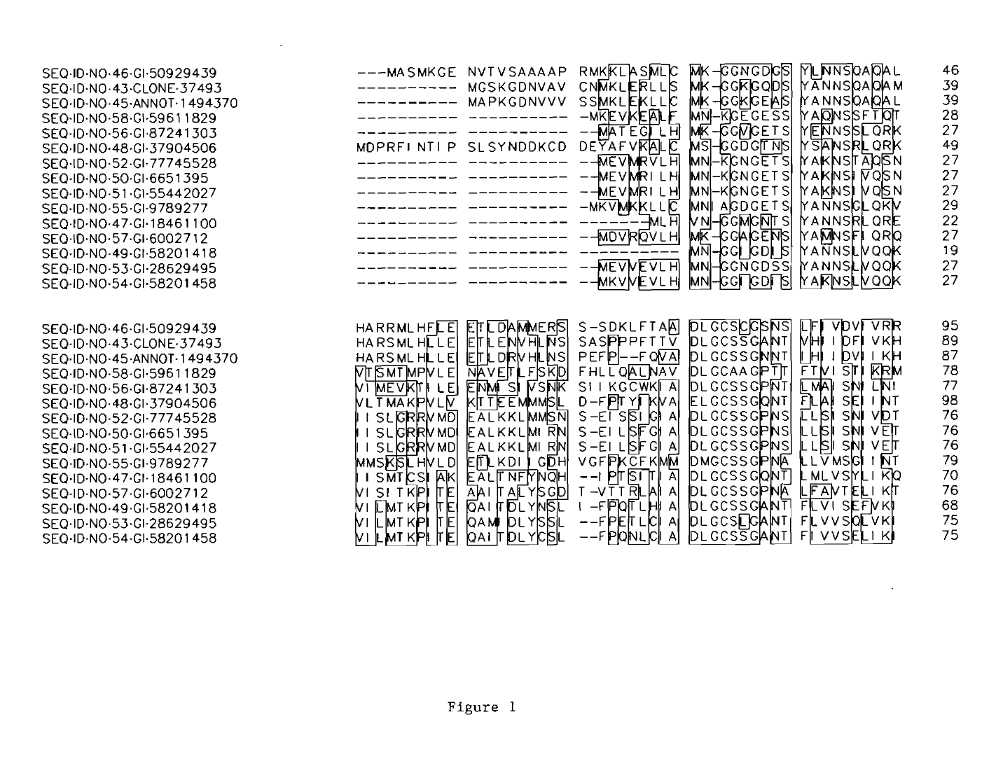

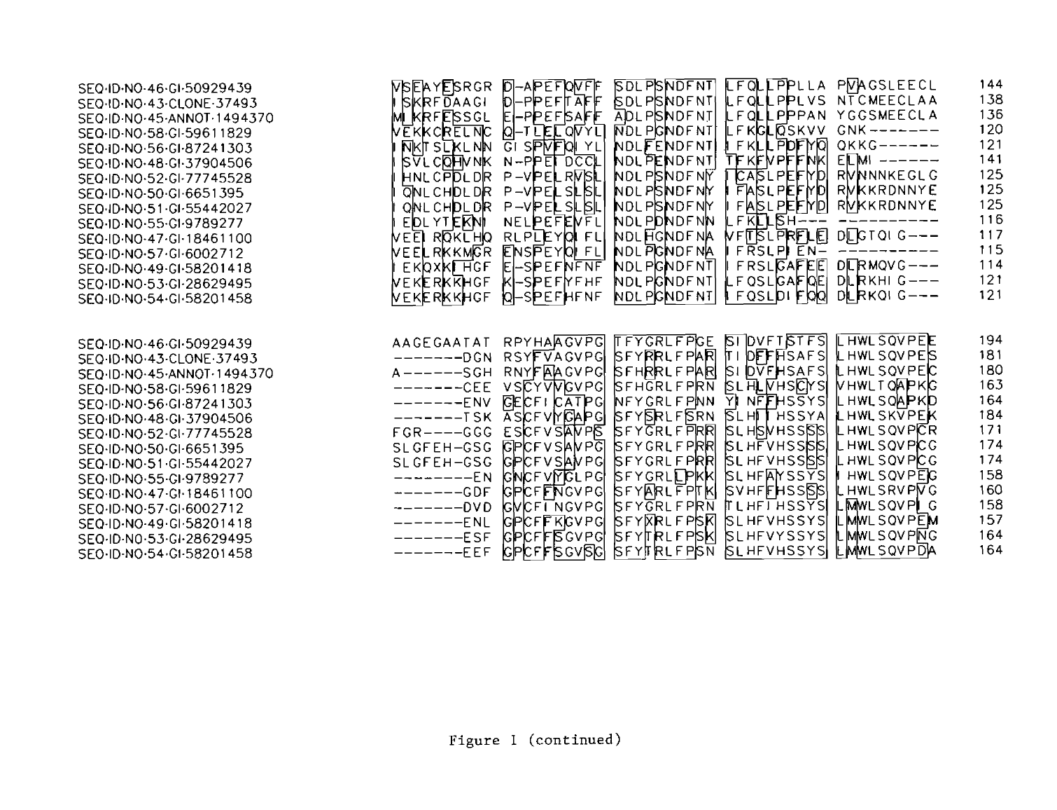

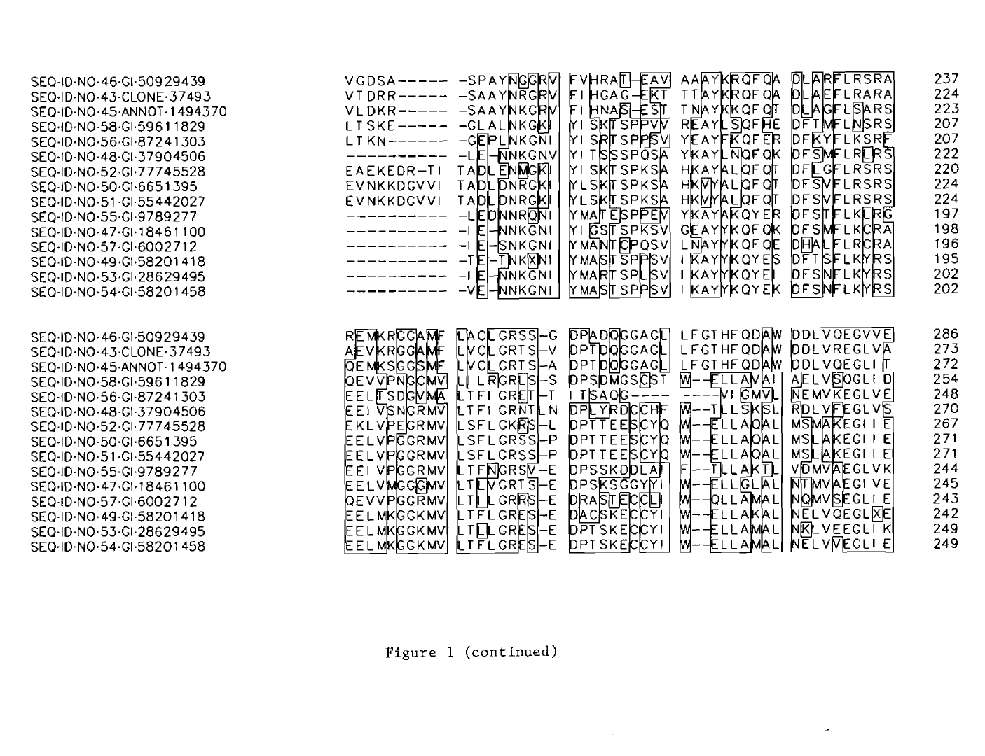

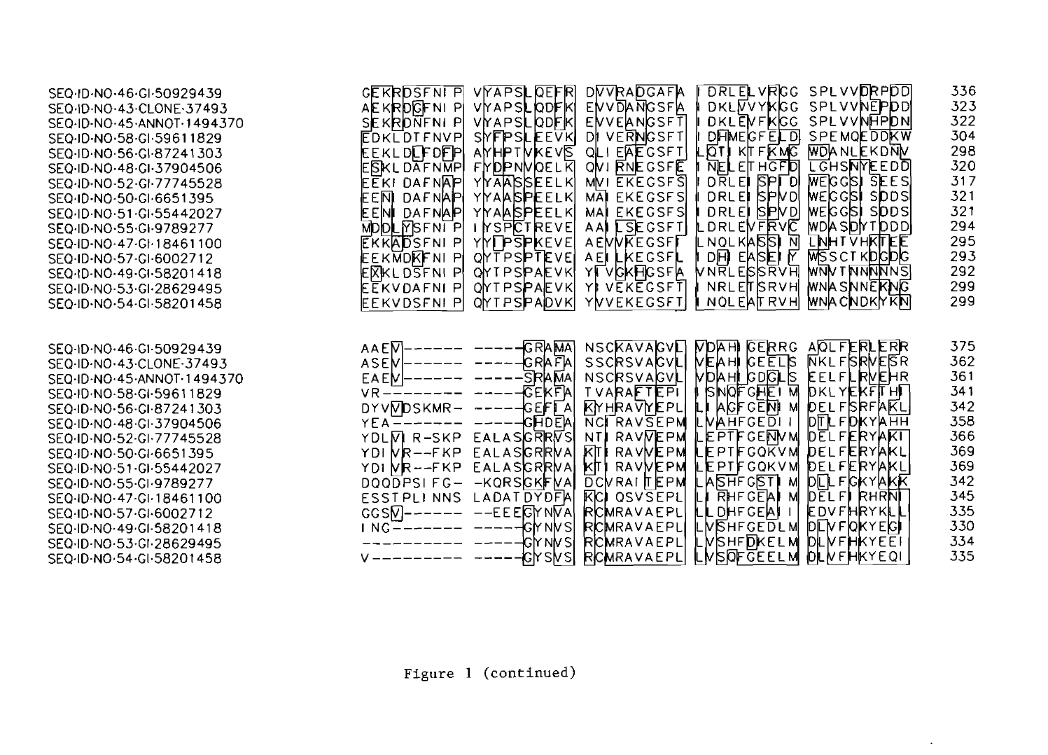

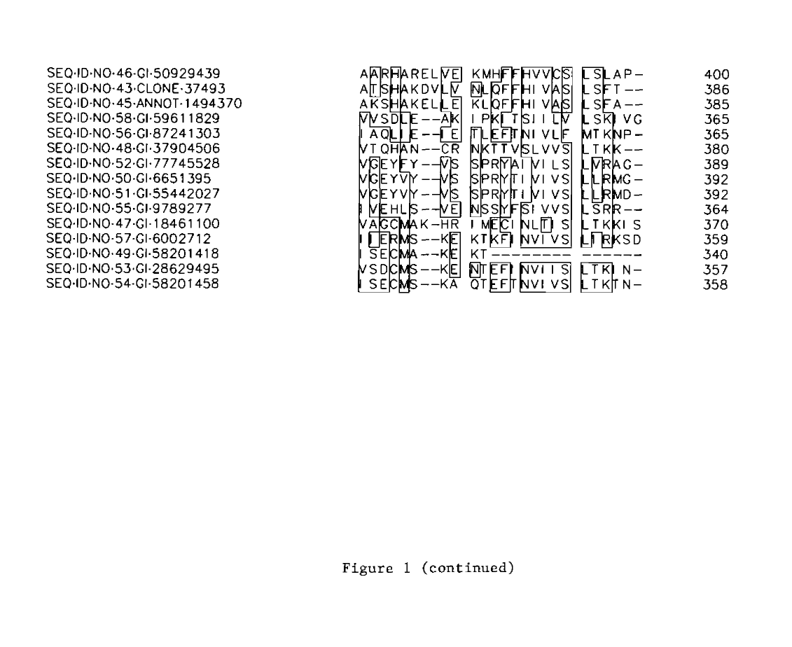

An FRE-tolerance polypeptide can comprise the amino acid sequence of Ceres Clone 37493 as set forth FIG. 1 and in SEQ ID NO: 43. Ceres Clone 37493 (SEQ ID NO: 43) is predicted to encode an S-adenosyl-L-methionine (SAM) dependent-salicylic acid carboxyl methyl transferase-like protein. Methyl transferases are a family of enzymes that catalyze the transfer of a methyl group from one molecule to another. S-adenosyl-L-methionine (SAM) dependent-salicylic acid carboxyl methyl transferase (SAMT) specifically catalyzes the formation of methylsalicylate from salicylic acid and the methyl donor, S-adenosyl-L-methionine (SAM).

Thus, an FRE-tolerance polypeptide can be an Arabidopsis polypeptide having the amino acid sequence set forth in SEQ ID NO: 43. Alternatively, an FRE-tolerance polypeptide can be a homolog, ortholog, or variant of the polypeptide having the amino acid sequence set forth in SEQ ID NO: 43. For example, an FRE-tolerance polypeptide can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO: 43.

Amino acid sequences of homologs and/or orthologs of the polypeptide having the amino acid sequence set forth in SEQ ID NO: 43 are provided in FIG. 1. For example, the alignment in FIG. 1 provides the amino acid sequences of Ceres Clone 37493 (SEQ ID NO: 43) and gi|50929439 (SEQ ID NO: 46), Ceres gDNA 1494370 (SEQ ID NO: 45), gi|59611829 (SEQ ID NO: 58), gi|87241303 (SEQ ID NO: 56), gi|37904506 (SEQ ID NO: 48), gi|77745528 (SEQ ID NO: 52), gi|6651395 (SEQ ID NO: 50), gi|55442027 (SEQ ID NO: 51), gi|9789277 (SEQ ID NO: 55), gi|18461100 (SEQ ID NO: 47), gi|6002712 (SEQ ID NO: 57), gi|58201418 (SEQ ID NO: 49), gi|28629495 (SEQ ID NO: 53), gi|58201458 (SEQ ID NO: 54).

In some cases, an FRE-tolerance polypeptide includes a polypeptide having at least 80% sequence identity, e.g., 80%, 85%, 90%, 95%, 97%, 98%, or 99% sequence identity, to an amino acid sequence corresponding to SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or SEQ ID NO: 58.

An FRE-tolerance polypeptide encoded by a recombinant nucleic acid can be a native FRE-tolerance polypeptide, i.e., one or more additional copies of the coding sequence for an FRE-tolerance polypeptide that is naturally present in the cell. Alternatively, an FRE-tolerance polypeptide can be heterologous to the cell, e.g., a transgenic Lycopersicon plant can contain the coding sequence for an FRE-tolerance polypeptide from a Glycine plant.

An FRE-tolerance polypeptide can include additional amino acids that are not involved in FRE-tolerance, and thus can be longer than would otherwise be the case. For example, an FRE-tolerance polypeptide can include an amino acid sequence that functions as a reporter. Such an FRE-tolerance polypeptide can be a fusion protein in which a green fluorescent protein (GFP) polypeptide is fused to, e.g., SEQ ID NO: 41, SEQ ID NO: 82, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or in which a yellow fluorescent protein (YFP) polypeptide is fused to, e.g., SEQ ID NO: 41, SEQ ID NO: 82, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58. In some embodiments, an FRE-tolerance polypeptide includes a purification tag, a chloroplast transit peptide, a mitochondrial transit peptide, or a leader sequence added to the amino or carboxy terminus.

FRE-tolerance polypeptide candidates suitable for use in the invention can be identified by analysis of nucleotide and polypeptide sequence alignments. For example, performing a query on a database of nucleotide or polypeptide sequences can identify homologs and/or orthologs of FRE-tolerance polypeptides. Sequence analysis can involve BLAST, Reciprocal BLAST, or PSI-BLAST analysis of nonredundant databases using known FRE-tolerance polypeptide amino acid sequences. Those polypeptides in the database that have greater than 40% sequence identity can be identified as candidates for further evaluation for suitability as an FRE-tolerance polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in FRE-tolerance polypeptides, e.g., conserved functional domains.

The identification of conserved regions in a template or subject polypeptide can facilitate production of variants of wild type FRE-tolerance polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within FRE-tolerance polypeptides. These conserved regions can be useful in identifying functionally similar (orthologous) FRE-tolerance polypeptides.

In some instances, suitable FRE-tolerance polypeptides can be synthesized on the basis of consensus functional domains and/or conserved regions in polypeptides that are homologous FRE-tolerance polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of FRE-tolerance polypeptides are shown in FIG. 1. Each FIGURE represents an alignment of the amino acid sequence of an FRE-tolerance polypeptide with the amino acid sequences of corresponding homologs and/or orthologs. Amino acid sequences of FRE-tolerance polypeptides and their corresponding homologs and/or orthologs have been aligned to identify conserved amino acids as shown in FIG. 1. A dash in an aligned sequence represents a gap, i.e., a lack of an amino acid at that position. Identical amino acids or conserved amino acid substitutions among aligned sequences are identified by boxes. Each conserved region contains a sequence of contiguous amino acid residues.

Useful polypeptides can be constructed based on the conserved regions in FIG. 1. Such a polypeptide includes the conserved regions, arranged in the order depicted in the FIGURE from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Conserved regions can be identified by homologous polypeptide sequence analysis as described above. The suitability of polypeptides for use as oil-modulating polypeptides can be evaluated by functional complementation studies.

Useful polypeptides can also be identified based on the polypeptides set forth in FIG. 1 using algorithms designated as Hidden Markov Models. A “Hidden Markov Model (HMM)” is a statistical model of a consensus sequence for a group of structurally and/or functionally related polypeptides. See, Durbin et al., Biological Sequence Analysis: Probabilistic Models of Proteins and Nucleic Acids, Cambridge University Press, Cambridge, UK (1998). An HMM is generated by the program HMMER 2.3.2 using the group of structurally and/or functionally related sequences as input and the default program parameters. HMMER 2.3.2 was released Oct. 3, 2003 under a GNU general public license, and is available from various sources on the World Wide Web such as hmmer.janelia.org, hmmer.wustl.edu, and fr.com/hmmer232/. The program outputs the model as a text file.

The HMM for a group of structurally and/or functionally related polypeptides can be used to determine the likelihood that a query subject polypeptide sequence is a better fit to that particular HMM than to a null HMM generated using a group of sequences that are not structurally or functionally related. The likelihood that a query subject polypeptide sequence is a better fit to an HMM than to a null HMM is indicated by the HMM bit score, a number generated when the query subject sequence and an HMM are input into the HMMER program, and the query subject is fitted to the HMM with HMMER configured for glocal alignments. A high HMM bit score also indicates a greater likelihood that the query subject sequence carries out one or more of the biochemical or physiological function(s) of the polypeptides used to generate the HMM. A high HMM bit score is at least 20, and often is higher.

An FRE-tolerance polypeptide can fit an HMM provided herein with an HMM bit score greater than 20 (e.g., greater than 30, 40, 50, 60, 70, 80, 90, 100, 200, 300, 400, 500, 600, 700, 800 or 900). In some embodiments, an FRE tolerance polypeptide can fit an HMM provided herein with an HMM bit score of 814, 821, 791, 847, 813, 797, 877, 877, 855, 892, 903, 822, 814, 851 or 800. In some cases, an FRE-tolerance polypeptide can fit an HMM provided herein with an HMM bit score that is about 50%, 60%, 70%, 80%, 90%, or 95% of the HMM bit score of any homologous and/or orthologous polypeptide provided in Table 11. In some cases, an FRE-tolerance polypeptide can fit an HMM described herein with an HMM bit score greater than 20, and can have a conserved domain, e.g., a PFAM domain, or a conserved region having 70% or greater sequence identity (e.g., 75%, 80%, 85%, 90%, 95%, or 100% sequence identity) to a conserved domain or region present in an FRE-tolerance polypeptide disclosed herein.

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to increase FRE-tolerance. A recombinant nucleic acid construct comprises a nucleic acid encoding an FRE-tolerance polypeptide as described herein, operably linked to a regulatory region suitable for expressing the FRE-tolerance polypeptide in the plant or cell. Thus, a nucleic acid can comprise a coding sequence that encodes any of the FRE-tolerance polypeptides as set forth in SEQ ID NO: 41 and SEQ ID NO: 43. In some cases, a recombinant nucleic acid construct can include a nucleic acid comprising less than the full-length coding sequence of an FRE-tolerance polypeptide. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given FRE-tolerance polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 10/950,321; PCT/US05/011105; PCT/US05/034308; and PCT/US05/23639. Nucleotide sequences of promoters are set forth in SEQ ID NOs: 1-39 and SEQ ID NOs: 59-67. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO: 37), YP0144 (SEQ ID NO: 20), YP0190 (SEQ ID NO: 23), p13879 (SEQ ID NO: 36), YP0050 (SEQ ID NO: 16), p32449 (SEQ ID NO: 38), 21876 (SEQ ID NO: 1), YP0158 (SEQ ID NO: 21), YP0214 (SEQ ID NO: 24), YP0380 (SEQ ID NO: 31), PT0848 (SEQ ID NO: 13), and PT0633 (SEQ ID NO: 5) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO: 3), PT0668 (SEQ ID NO: 2), PT0886 (SEQ ID NO: 15), YP0144 (SEQ ID NO: 20), YP0380 (SEQ ID NO: 70), and PT0585 (SEQ ID NO: 4).

Vascular Tissue Promoters

Examples of promoters that have high or preferential activity in vascular bundles include YP0087 (SEQ ID NO: 62), YP0093 (SEQ ID NO: 63), YP0108 (SEQ ID NO: 103), YP0022 (SEQ ID NO: 61), and YP0080 (SEQ ID NO: 67). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO: 31), PT0848 (SEQ ID NO: 13), YP0381 (SEQ ID NO: 32), YP0337 (SEQ ID NO: 27), PT0633 (SEQ ID NO: 5), YP0374 (SEQ ID NO: 29), PT0710 (SEQ ID NO: 9), YP0356 (SEQ ID NO: 28), YP0385 (SEQ ID NO: 34), YP0396 (SEQ ID NO: 35), YP0388 (SEQ ID NO: 65), YP0384 (SEQ ID NO: 33), PT0688 (SEQ ID NO: 8), YP0286 (SEQ ID NO: 26), YP0377 (SEQ ID NO: 30), PD1367 (SEQ ID NO: 39), PD0901 (SEQ ID NO: 60), and PD0898 (SEQ ID NO: 59). Examples of nitrogen-inducible promoters include PT0863 (SEQ ID NO: 14), PT0829 (SEQ ID NO: 12), PT0665 (SEQ ID NO: 6), and PT0886 (SEQ ID NO: 15). Examples of shade-inducible promoters include PR0924 (SEQ ID NO: 66), and PT0678 (SEQ ID NO: 7).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential, such as PT0678 (SEQ ID NO: 7), and senescence-preferential promoters. Promoters designated YP0086 (SEQ ID NO: 17), YP0188 (SEQ ID NO: 22), YP0263 (SEQ ID NO: 25), PT0758 (SEQ ID NO: 11), PT0743 (SEQ ID NO: 10), PT0829 (SEQ ID NO: 12), YP0119 (SEQ ID NO: 19), and YP0096 (SEQ ID NO: 18), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding an FRE-tolerance polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct. In some embodiments, transgenic plants exhibiting a desired trait are selected from among independent transformation events.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous FRE-tolerance polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

Plant Species

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, foxglove, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, peach, pear, peas, pecan, pepper, pistachio, plum, poplar, poppy, potato, pumpkin, oilseed rape, quinoa, rapeseed (high erucic acid and canola), safflower, sesame, soaptree bark, soybean, spinach, strawberry, sugar beet, sunflower, sweet potatoes, tea, tomato, walnut, and yams, as well as monocots such as banana, barley, bluegrass, coconut, corn, date palm, fescue, field corn, garlic, millet, oat, oil palm, onion, palm kernel oil, pineapple, popcorn, rice, rye, ryegrass, sorghum, sudangrass, sugarcane, sweet corn, switchgrass, turf grasses, timothy, and wheat. Gymnosperms such as fir, pine, and spruce can also be suitable.

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malpighiales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violates. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ephedrales, Ginkgoales, Gnetales, and Pinales.

The methods and compositions can be used over a broad range of plant species, including species from the dicot genera Acokanthera, Aconitum, Aesculus, Alangium, Alchornea, Alexa, Alseodaphne, Amaranthus, Ammodendron, Anabasis, Anacardium, Angophora, Anisodus, Apium, Apocynum, Arabidopsis, Arachis, Argemone, Asclepias, Atropa, Azadirachta, Beilschmiedia, Berberis, Bertholletia, Beta, Betula, Bixa, Bleekeria, Borago, Brassica, Calendula, Camellia, Camptotheca, Canarium, Cannabis, Capsicum, Carthamus, Carya, Catharanthus, Centella, Cephaelis, Chelidonium, Chenopodium, Chrysanthemum, Cicer, Cichorium, Cinchona, Cinnamomum, Cissampelos, Citrus, Citrullus, Cocculus, Cocos, Coffea, Cola, Convolvulus, Coptis, Corylus, Corymbia, Crambe, Crotalaria, Croton, Cucumis, Cucurbita, Cuphea, Cytisus, Datura, Daucus, Dendromecon, Dianthus, Dichroa, Digitalis, Dioscorea, Duguetia, Erythroxylum, Eschscholzia, Eucalyptus, Euphorbia, Euphoria, Ficus, Fragaria, Galega, Gelsemium, Glaucium, Glycine, Glycyrrhiza, Gossypium, Helianthus, Heliotropium, Hemsleya, Hevea, Hydrastis, Hyoscyamus, Jatropha, Juglans, Lactuca, Landolphia, Lavandula, Lens, Linum, Litsea, Lobelia, Luffa, Lupinus, Lycopersicon, Macadamia, Mahonia, Majorana, Malus, Mangifera, Manihot, Meconopsis, Medicago, Menispermum, Mentha, Micropus, Nicotiana, Ocimum, Olea, Origanum, Papaver, Parthenium, Persea, Petunia, Phaseolus, Physostigma, Pilocarpus, Pistacia, Pisum, Populus, Prunus, Psychotria, Pyrus, Quillaja, Rabdosia, Raphanus, Rhizocarya, Ricinus, Rosa, Rosmarinus, Rubus, Rubia, Salix, Salvia, Sanguinaria, Scopolia, Senecio, Sesamum, Simmondsia, Sinapis, Sinomenium, Solanum, Sophora, Spinacia, Stephania, Strophanthus, Strychnos, Tagetes, Theobroma, Thymus, Trifolium, Trigonella, Vaccinium, Vicia, Vigna, Vinca, and Vitis; and the monocot genera Agrostis, Allium, Ananas, Andropogon, Areca, Asparagus, Avena, Cocos, Colchicum, Convallaria, Curcuma, Cynodon, Elaeis, Eragrostis, Festuca, Festulolium, Galanthus, Hemerocallis, Hordeum, Lemna, Lolium, Musa, Oryza, Panicum, Pennisetum, Phleum, Phoenix, Poa, Ruscus, Saccharum, Secale, Sorghum, Triticosecale, Triticum, Veratrum, Zea, and Zoysia; and the gymnosperm genera Abies, Cephalotaxus, Cunninghamia, Ephedra, Picea, Pinus, Populus, and Pseudotsuga.

In some embodiments, a plant can be from a species selected from Avena sativa, Brassica spp., Brassica napus, Brassica rapa, Brassica oleracea, Glycine max, Gossypium spp., Gossypium hirsutum, Gossypium herbaceum, Helianthus annuus, Hordeum vulgare, Lactuca sativa, Medicago sativa, Oryza sativa, Panicum virgatum, Secale cereale, Triticum aestivum, and Zea mays.

### Transgenic Plant Phenotypes

Selection or screening can be carried out among a population of transformed cell, callus, tissue, or plant material to identify transformants using selectable marker genes such as herbicide resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of a polypeptide described herein. For example, selection and/or screening can be carried out to identify those transgenic plants having a statistically significant difference in a response to far-red-enriched (FRE) light conditions relative to a control plant that lacks the transgene. Selection and/or screening can be carried out over one or more generations to identify those plants that have the desired trait. Selection and/or screening can also be carried out in more than one geographic location if desired. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is expected to be exhibited by the plant.

Transgenic plants can have an altered phenotype as compared to a corresponding control plant that either lacks the transgene or does not express the transgene. A polypeptide can affect the phenotype of a plant (e.g., a transgenic plant) when expressed in the plant, e.g., at the appropriate time(s), in the appropriate tissue(s), or at the appropriate expression levels. Phenotypic effects can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

The phenotype of a transgenic plant and a corresponding control plant that either lacks the transgene or does not express the transgene can be evaluated under particular environmental conditions. For example, far-red enriched (FRE) light conditions are a useful system for simulating shade. Red wavelengths typically range from a photon irradiance of about 630 to a photon irradiance of about 700 nm. Far-red wavelengths typically range from a photon irradiance of about 700 to a photon irradiance of about 750 nm. The ratio of red:far-red (R:FR) light perceived by plant phytochromes, a family of specialized information-transducing molecules that plants rely upon to monitor changes in the quantity, quality and direction of light, is important in determining whether plants display shade avoidance phenotypes. Typically, a red/far-red (R:FR) ratio of <1 results in shade avoidance responses.

Far-red light enriched (FRE) conditions are conditions in which the ratio of the fluence at 633 nm to the fluence at 740 nm (red/far-red) is less than 1, e.g., less than about 0.9, less than about 0.8, less than about 0.7, less than about 0.6, less than about 0.5, less than about 0.4, less than about 0.3, less than about 0.2, less than about 0.1, or less than about 0.05. The phenotype of a transgenic plant can be assayed under FRE conditions.

In some embodiments, the phenotype of a transgenic plant is assayed under FRE conditions in which there is continuous FRE light during the light period of a light/dark cycle, that is, growth conditions in which any and all incoming light received by a plant has an R:FR ratio of less than 1.0. Continuous FRE conditions can be, for example, 16 hours of FRE at a red:far-red ratio of 0.22 with the following overall fluence rates: blue₄₅₀=10.88 μmol/m²/s, red₆₃₃=32.9 μmol/m²/s, far-red₇₄₀=148.5 μmol/m²/s, PPFD₄₀₀₋₇₀₀=62.25 μmol/m²/s, alternating with 8 hours of darkness.

In some embodiments, the phenotype of a transgenic plant is assayed under FRE conditions in which there is a pulse of FRE light during the light period of a light/dark cycle. For example, plants can be grown under light having a red:far-red ratio of >1.0 and then exposed to a pulse of FRE light. The FRE pulse can be for 0.1, 0.2, 0.5, 1.0, 1.5, 2.0, 2.5, 3.0, 4.0, 5.0, 6.0, 7.0, 8.0 hours or more. The FRE pulse can take place at any time during a 24 hour day, e.g., at the beginning, middle, or end of the period of light exposure. One suitable set of conditions for a pulse of FRE light is End-of Day-Far-red Enriched (EODFR) conditions. EODFR conditions can be 9.5 hours light with a red:far-red ratio of >1.0, followed by a 30 minute pulse of far-red light at the end of each light cycle, alternating with 14 hours of darkness. The light cycle can have a red:far-red ratio of about 5.5, with the following fluence rates: blue₄₅₀=12 μmol/m²/s, red₆₃₃=22 μmol/m²/s, far-red₇₄₀=4 μmol/m²/s, PPFD₄₀₀₋₇₀₀=55 μmol/m²/s; the far-red pulse can have a red:far-red ratio of about 0.14 with the fluence rates: blue₄₅₀=0.004 μmol/m²/s, red₆₃₃=10 μmol/m²/s, far-red₇₄₀=70 μmol/m²/s, PPFD₄₀₀₋₇₀₀=8 μmol/m²/s. Sources of lighting equipment appropriate for producing and maintaining FRE conditions are known to those in art.

The phenotypes of a transgenic plant and a corresponding control plant that either lacks the transgene or does not express the transgene can be evaluated under FRE conditions. Phenotypic responses by plants in response to reduction in the R:FR ratio of incoming light can typically include increases in extension growth, e.g., increased petiole length, increased hypocotyl length, increased internode spacing, and increased leaf elongation in cereals; retardation in leaf development, e.g., reduced leaf thickness and reduced leaf area growth; increased apical dominance, e.g., inhibition of branching and tillering; retarded chloroplast development, e.g., reduced chlorophyll synthesis and a change in the balance of the chlorophyll a:b ratio; alterations in flowering and seed/fruit production, e.g., an increased rate of flowering, a reduction in seed set, and truncation of fruit development; and a reduction in storage organ deposition. A transgenic plant expressing an FRE-tolerance polypeptide can exhibit one or more of the following phenotypes under FRE conditions relative to a corresponding control plant that either lacks the transgene or does not express the transgene: reduction in extension growth, e.g., shorter petiole length, shorter hypocotyl length, reduced internode spacing, and reduced leaf elongation in cereals; accelerated in leaf development, e.g., increased leaf thickness and increased leaf area growth; reduced apical dominance, e.g., no inhibition of branching and tillering; accelerated chloroplast development, e.g., increased chlorophyll synthesis and no change in the balance of the chlorophyll a:b ratio; no alterations in flowering and seed/fruit production, e.g., no increased rate of flowering, no reduction in seed set, and no truncation of fruit development; and no reduction in storage organ deposition.

Typically, a difference (e.g., an increase) in a morphological feature in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the dimensions of any individual morphological feature is statistically significant at p<0.01, p<0.005, or p<0.001. A statistically significant difference in, for example, a morphological feature in a transgenic plant compared to the corresponding morphological feature a control plant indicates that (1) expression of the recombinant nucleic acid present in the transgenic plant confers the alteration in the morphological feature and/or (2) the recombinant nucleic acid warrants further study as a candidate for altering the morphological feature in a plant.

One suitable phenotype to measure is petiole length. When wild-type seedlings are grown under FRE conditions, the petiole length is typically significantly increased relative to the petiole length found in wild-type seedlings grown under non-FRE conditions. Thus, seedlings of a transgenic plant and seedlings of a corresponding control plant that either lacks the transgene or does not express the transgene can be grown under FRE conditions and at the appropriate time, petiole lengths from seedlings of each group can be measured. Under FRE conditions, a seedling in which the expression of an FRE-tolerance polypeptide is increased can have a statistically significantly shorter petiole length than a seedling of a corresponding control plant that either lacks the transgene or does not express the transgene.

In some embodiments, under FRE conditions, a seedling in which expression of an FRE-tolerance polypeptide is increased can have a shorter petiole length relative to the corresponding control seedlings that either lack the transgene or do not express the transgene. The petiole length can be shorter by at least 20 percent, e.g., 20, 25, 30, 35, 40, 45, 50, 55, 60, 65, 70, or 75 percent, as compared to the petiole length in a corresponding control plant that does not express the transgene.

Another suitable phenotype to measure is hypocotyl length. When wild-type seedlings are grown under EODFR conditions, the hypocotyl length is typically significantly increased relative to the hypocotyl length found in wild-type seedlings grown under control light conditions. Thus, seedlings of a transgenic plant and seedlings of a corresponding control plant that either lacks the transgene or does not express the transgene can be grown under EODFR conditions and at the appropriate time, hypocotyl lengths from seedlings of each group can be measured. Under EODFR conditions, a seedling in which the expression of an EODFR-tolerance polypeptide is increased can have a statistically significantly shorter hypocotyl length than a seedling of a corresponding control plant that either lacks the transgene or does not express the transgene.

In some embodiments, under EODFR conditions, a seedling in which expression of an EODFR-tolerance polypeptide is increased can have a shorter hypocotyl length relative to the corresponding control seedlings that either lack the transgene or do not express the transgene. The hypocotyl length can be shorter by at least 20 percent, e.g., 20, 25, 30, 35, 40, 45, 50, 55, 60, 65, 70, 75, or 80 percent, as compared to the hypocotyl length in a corresponding control plant that does not express the transgene.

Transgenic plants provided herein have particular uses in agricultural industries. For example, transgenic plants expressing an FRE-tolerance polypeptide provided herein can maintain development and maturation of such plants under shade conditions, compared to a corresponding control plant. Such a trait can increase plant survival and seedling establishment at high density plant populations in crops even when plants are near mature growth stages. Transgenic plants expressing an FRE-tolerance polypeptide can be more densely planted than those that are not FRE-tolerant. Expression of an FRE tolerance polypeptide in crop plants can provide increased yields of seed and non-seed tissues from such plants compared to non-FRE tolerant plants grown under the same conditions.

Information that the polypeptides disclosed herein can increase FRE-tolerance can be useful in breeding of crop plants. Based on the effect of disclosed polypeptides on FRE-tolerance, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in FRE-tolerance. Those polymorphisms that are correlated with an alteration in FRE-tolerance can be incorporated into a marker assisted breeding program to facilitate the development of lines that have a desired alteration in FRE-tolerance. Typically, a polymorphism identified in such a manner is used with polymorphisms at other loci that are also correlated with a desired alteration in FRE-tolerance.

### Articles of Manufacture

Seeds of transgenic plants described herein can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package. The package label may indicate that the seed herein incorporates transgenes that provide improved response to shade conditions.

The invention will be further described in the following examples, which do not limit the scope of the invention described in the claims.

### EXAMPLES

### Example 1

### Transgenic Plants

The following symbols are used in the Examples: T1: first generation transformant; T2: second generation, progeny of self-pollinated T1 plants; T3: third generation, progeny of self-pollinated T2 plants. Independent transformations are referred to as events.

The following nucleic acids were isolated from Arabidopsis thaliana plants. Ceres Clone 258241 (SEQ ID NO: 40) is a cDNA clone that is predicted to encode a putative zinc finger transcription factor protein. Ceres Clone 37493 (SEQ ID NO: 42) is a cDNA clone that is predicted to encode an S-adenosyl-L-methionine (SAM) dependent-salicylic acid carboxyl methyl transferase-like protein.

Each isolated nucleic acid described above was cloned, using standard molecular biology techniques, into a Ti plasmid vector, CRS 338, which encodes a selectable marker gene, phosphinothricin acetyltransferase, that confers Finale™ resistance on transformed plants. Constructs were made using the CRS 338 vector that contained either the cDNA Ceres Clone 258241 (SEQ ID NO: 40) and or the cDNA Ceres Clone 37493 (SEQ ID NO: 42) operably linked in the sense orientation relative to a CaMV 35S constitutive promoter.

The constructs were introduced separately into Arabidopsis ecotype Wassilewskija (WS-2) plants by the floral dip method essentially as described in Bechtold, N. et al., C.R. Acad. Sci. Paris, 316:1194-1199 (1993). Two independent transformations were carried out with the CRS 338 construct encoding Ceres Clone 258241 (SEQ ID NO: 40), resulting in two independent events designated ME01990 and ME01593. A single transformation was carried out with the CRS 338 construct encoding Ceres Clone 37493 (SEQ ID NO: 42), resulting in an event designated ME03531. The presence of the vector DNA in each of these events was confirmed by screening the T1 plants for Finale™ resistance. The presence of Ceres Clone DNA in the T1 plants was confirmed by PCR amplification of insert sequences in DNA extracted from green leaf tissue and the identity of the Ceres Clone was determined by sequencing of the PCR products. Control plants were transformed with the CRS vector lacking inserted Arabidopsis cDNA. T1 plants were evaluated for morphology and development. Certain plants from ME01990 events exhibited larger plant size, larger rosette area, more rosette leaves, taller, thicker inflorescences and delayed flowering time. The morphology and development of T1 plants from ME01593 events was similar to that of control plants. Certain plants from ME03531 events exhibited smaller plant size, smaller rosette area, curled leaves, dark green color, and reduced fertility when compared to control plants.

Plants from these independently transformed events were evaluated for their qualitative phenotype according to the methods described in Examples 2 and 3 below. Plants that were attenuated in their shade avoidance response in the T1 generation, i.e., plants that had reduced petiole or hypocotyl length in response to Far-red Enriched Assay (FRE) conditions or End-of Day-Far-red (EODFR) Assay conditions, respectively, were selected. T1 seeds were germinated and allowed to self-pollinate. T2 seeds were collected and a portion was germinated, allowed to self-pollinate, and T3 seeds were collected.

In the T2 and T3 generations, the co-segregation of phenotype and transgene was analyzed. In all the ME lines tested, the reduced petiole or hypocotyl length in response to FRE or EODFR conditions, respectively, segregated at a ratio of 3:1. Chi-square analysis showed that there was a statistically significant correlation between the reduced petiole and/or hypocotyl length phenotype and the presence of the transgene in the T2 and T3 progeny.

### Example 2

### Far-Red Enriched (FRE) Assay

A Far-red Enriched (FRE) Assay was carried out on seedlings in order to evaluate the effect of FRE conditions on petiole length. For the Far-red Enriched Assay, seeds were plated on 1.0% sucrose, 0.5×MS media (PhytoTech) agar plates, cold-treated for 3-4 days at 4° C., then germinated and grown for 5 days under cycling white light. Cycling white light conditions were 16 hours of light alternating with 8 hours of darkness at 60 μmol/m²/s generated by a T12 rapid start fluorescent lamp and four 32W 4100K Ecologic fluorescent light bulbs (Octron, Sylvania). Seedlings were then maintained for an additional 7 days under Far-red Enriched conditions. Under FRE conditions, the cycling white light conditions of 16 hours of light alternating with 8 hours of darkness at 60 μmol/m²/s were supplemented with far-red light provided by a SNAP-LITE far-red light box Red/Far-Red (Quantum devices, SL1515-670-735). The overall fluence rates under these conditions were: blue₄₅₀=10.88 μmol/m²/s, red₆₃₃=32.9 μmol/m²/s, far-red₇₄₀=148.5 μmol/m²/s, PPFD₄₀₀₋₇₀₀=62.25 μmol/m²/s; the red:far-red ratio was 0.22. Control seedlings were not shifted to FRE conditions, but instead were maintained for 7 days under cycling white light conditions of 16 hours of light alternating with 8 hours of darkness at ˜60 μmol/m²/s. Petiole lengths were measured 12 days after germination.

### Example 3

### End-of-Day-Far-Red (EODFR) Assay

An End-of-Day-Far-red (EODFR) Assay was carried out on seedlings in order to evaluate the effect of EODFR conditions on hypocotyl length. For the EODFR Assay, seeds were plated on 0.5% sucrose, 1×MS media (PhytoTech) agar plates, cold-treated for 3-4 days at 4° C., then germinated for 2 days under continuous white light at about 60 μmol/m²/s in walk-in Conviron growth chambers. Seedlings were then exposed to EODFR conditions for 2 days. EODFR conditions were 9.5 hours light, followed by a 30 minute pulse of far-red light at the end of each light cycle, alternating with 14 hours of darkness. Two Gro-Lux (Sylvania, 24660) and two Cool White (Phillips) lights at about 60 μmol/m²/s PPFD, with a red:far-red ratio of about 5.5, were used for the light cycle; the fluence rates under these conditions were: blue₄₅₀=12 μmol/m²/s, red₆₃₃=22 μmol/m²/s, far-red₇₄₀=4 μmol/m²/s, PPFD₄₀₀₋₇₀₀=55 μmol/m²/s. The far-red pulse was generated by 3 SNAP-LITE Far-red light boxes (Quantum devices, SL1515-670-735) at about 8 μmol/m²/s PPFD, with a red:far-red ratio of about 0.14; the fluence rates under these conditions were: blue₄₅₀=0.004 μmol/m²/s, red₆₃₃=10 μmol/m²/s, far-red₇₄₀=70 μmol/m²/s, PPFD₄₀₀₋₇₀₀=8 μmol/m²/s. Control seedlings were cultured exactly as above except that they did not receive the far-red pulse; that is, following germination, they were exposed for two days to a cycle of 10 hours of light alternating with 14 hours of darkness under 2 Gro-Lux and 2 Cool white lights at about 60 μmol/m²/s PPFD, with a red:far-red ratio of about 5.5. Plates were rotated on the third day after plating and hypocotyl length was measured on the fourth day after plating.

### Example 4

### Foliar Canopy Petiole Assay

Foliar canopy petiole assays were carried out by germinating Arabidopsis seeds in the dark for 4 days at 4 C on MS agar plates, followed by 5 days at room temperature with a 16 hr light/8 hr dark cycle, 250 μmol/m2·s with a red:far red ratio of about 4.2. Plates with germinated seedlings were then transferred to a greenhouse where they were placed between pots of tobacco plants that were approximately six weeks old, and grown under a 16 hr light/8 hr dark cycle, 2.1-5.6 μmol/m2·s with a red:far red ratio of <0.3. After 7 days under the tobacco canopy (12 days after germination), petiole length was measured. Plates with control seedlings were maintained at 16 hr light/8 hr dark cycle, 250 μmol/m2·s with a red:far red ratio of about 4.2 instead of being transferred to the tobacco canopy.

### Example 5

### Analysis of ME01593 Events

The effect of FRE conditions on petiole length in ME01593 T2 and T3 seedlings was evaluated using the FRE assay described in Example 2. Control plants for this experiment were wild-type WS-2 plants and ME01593 T2 and T3 segregating progeny that did not contain the Ceres Clone 258241 (SEQ ID NO: 40) putative zinc-finger transgene. These segregating progeny are referred to as Internal Controls. The T2 analysis included events ME01593-04 and ME01593-10. The T3 analysis included events ME01593-04-028 and ME01593-10-35; for the T3 analysis, segregating progeny of ME01593-10-6 were used as an Internal Control. Replicate plates of seeds from each of the ME events and control plants were germinated and grown under either FRE conditions or control light conditions as described in Example 2.

Results of assays of T2 seedlings are shown in Table 1. Under the FRE conditions in this experiment, both T2 wild type seedlings and T2 Internal Control seedlings showed an average increase of about 4 mm in petiole length relative to the petiole length observed in T2 wild-type and Internal Control seedlings grown under control light conditions. In contrast, the petiole length of T2 seedlings from ME01593-04 and ME01593-10 events was not increased under FRE conditions. Under FRE conditions, the petiole length of T2 seedlings from ME01593-04 and ME01593-10 events was statistically significantly shorter than the petiole length of T2 seedlings of wild-type and Internal Controls.

Results of assays of T3 seedlings are shown in Table 2. The petiole length of seedlings from the T3 events, ME01593-04-028 and ME01593-10-35, also showed no increase under FRE conditions relative to that of wild type seedlings and Internal Control seedlings. As shown in Table 2, seedlings from the ME01593-04-28 and the ME01593-10-35 events had a statistically significantly shorter petiole length in the T3 generation under FRE conditions as compared to the petiole length observed in wild-type and Internal Control seedlings grown under FRE conditions.

TABLE 1
Petiole length (mm) in ME01593 T2 seedlings

| | | | ME01593- | ME01593- | |
|---|---|---|---|---|---|
| Growth | ME01593- | ME01593- | 04 Internal | 10 Internal | Wild |
| conditions | 04 | 10 | Control | Control | type |
| FRE | 4.4^{a} | 4.4^{a} | 8.4 | 8.1 | 8.7 |
| Control | 4.4 | 4.4 | not | not | 4.7 |
| | | | determined | determined | |

^{a}Statistically significantly different from wild type at p < 0.05, based on a two-tailed Student's t-test.

TABLE 2
Petiole length (mm) in ME01593 T3 seedlings

| | | | ME01593-10 | |
|---|---|---|---|---|
| Growth | ME01593- | ME01593- | Internal | Wild |
| conditions | 04-28 | 10-35 | Control | type |
| FRE | 4.7^{a} | 4.4^{a} | 7.9 | 8.7 |
| Control | 4.1 | 4.0 | 4.6 | 4.7 |

^{a}Statistically significantly different from wild type at p < 0.05, based on a two-tailed Student's t-test.

The ME01593 events were also evaluated in the EODFR assay. No statistically significant differences in hypocotyl length were observed between the ME01593 events and wild-type WS-2 seedlings.

### Example 6

### Analysis of ME01990 Events

The effect of FRE conditions on petiole length in ME01990 T2 seedlings was evaluated using the FRE assay described in Example 2. Control plants for this experiment were wild-type plants and T2 segregating progeny that did not contain the Ceres Clone 258241 (SEQ ID NO: 40) putative zinc-finger transgene. These segregating progeny are referred to as Internal Controls. The T2 analysis included events ME01990-02 and ME01990-03. Replicate plates of seeds from each of the ME events and control plants were germinated and grown under either FRE conditions or control light conditions as described in Example 2.

Results of assays of T2 seedlings are shown in Table 3. Under the FRE conditions in this experiment, both T2 wild type seedlings and T2 Internal Control seedlings showed an average increase of about 4 mm in petiole length relative to the petiole length observed in T2 wild-type seedlings grown under control light conditions. In contrast, the petiole length of T2 seedlings from ME01990-02 and ME01990-03 events was not increased under FRE conditions. Under FRE conditions, the petiole length of T2 seedlings from ME01990-02 and ME01990-03 events was statistically significantly shorter than the petiole length of T2 seedlings of wild-type and Internal Controls.

ME01990 events were also evaluated in the EODFR assay. No statistically significant differences in hypocotyl length were observed between the ME01990 seedlings and T2 segregating progeny that did not contain the Ceres Clone 258241 (SEQ ID NO: 40) putative zinc-finger transgene.

TABLE 3
Petiole length (mm) in ME01990 T2 seedlings

| | | | ME01990- | ME01990- | |
|---|---|---|---|---|---|
| Growth | ME01990- | ME01990- | 02 Internal | 03 Internal | Wild |
| conditions | 02 | 03 | Control | Control | type |
| FRE | 5.2a | 4.5a | 9.6 | 7.5 | 8.7 |
| Control | 4.9 | 4.2 | not | not | 4.7 |
| | | | determined | determined | |

a= Statistically significantly different from wild type at p < 0.05, based on a two-tailed Student's t-test.

### Example 7

### Expression of mRNA Encoding Ceres Clone 258241 Protein Under FRE Conditions

Endogenous expression of mRNA encoding the Ceres Clone 258241 (SEQ ID NO: 41) putative zinc-finger protein was analyzed in wild-type Arabidopsis ecotype Wassilewskija (WS) seedlings. Replicate plates of seedlings were cultured as described in Example 2 for 7 days under control light conditions and then exposed to FRE conditions for either 1 or 24 hours. Control seedlings were maintained under control light conditions. At the end of the FRE exposure period, seedlings were harvested, RNA was extracted from whole seedlings and levels of specific mRNAs were assayed by RT-PCR. One aliquot of RNA in each sample was analyzed with Ceres Clone 258241 (SEQ ID NO: 41) putative zinc-finger protein specific primers. A second aliquot of each sample was analyzed with tubulin-specific primers; tubulin mRNA served as an internal control for total mRNA levels in each reaction. RT-PCR products were resolved by agarose gel electrophoresis.

In mRNA extracted from seedlings cultured under control light conditions for either 1 or 24 hours, the Ceres Clone 258241 (SEQ ID NO: 41) putative zinc-finger protein-specific primers generated similar amounts of the predicted amplicon. In contrast, no detectable Ceres Clone 258241 (SEQ ID NO: 41) putative zinc-finger protein-specific amplicon was present the mRNA extracted from seedlings exposed to either a 1 or 24 hour pulse of FRE conditions. Parallel reactions analyzed with the tubulin-specific primers generated similar levels of the tubulin-specific RT-PCR product in all samples, indicating that the samples obtained from plants grown under FRE conditions and those grown under control light conditions contained roughly equivalent amounts of total mRNA. These data indicated that the mRNA encoding the Ceres Clone 258241 (SEQ ID NO: 41) putative zinc finger protein is specifically and rapidly repressed under FRE conditions.

### Example 8

### Analysis of ME03531 Events

The effect of EODFR conditions on hypocotyl length in ME03531 T2 seedlings was evaluated using the EODFR assay described in Example 3. Internal control plants for this experiment were wild-type plants and T2 segregating progeny that did not contain the Ceres Clone 37493 (SEQ ID NO: 42) SAM dependent-carboxyl methyl transferase-like protein transgene. The T2 analysis included events ME03531-01, ME03531-02, and ME03531-07. Replicate plates of seeds from each of the ME events and control plants were germinated and grown under either EODFR conditions or control light conditions as described in Example 3. After 4 days of culture, hypocotyl length was measured using standard methods.

Results of assays of T2 seedlings are shown in Table 4. Under the EODFR conditions in this experiment, both T2 wild type seedlings and T2 Internal Control seedlings showed an average increase of about 2 mm in hypocotyl length relative to the hypocotyl length observed in T2 wild-type and Internal Control seedlings grown under control light conditions. In contrast, the hypocotyl length of T2 seedlings from ME03531-01, ME03531-02, and ME03531-07 events was not increased under EODFR conditions. Under EODFR conditions, the hypocotyl length of T2 seedlings from ME03531-01, ME03531-02, and ME03531-07 events was statistically significantly less than the hypocotyl length of T2 seedlings of wild-type and Internal Controls.

TABLE 4
Hypocotyl length (mm) in ME03531 T2 seedlings

| | | | | ME03531- | ME03531- | ME03531- | |
|---|---|---|---|---|---|---|---|
| | | | | 01 | 02 | 07 | |
| Growth | ME03531- | ME03531- | ME03531- | Internal | Internal | Internal | Wild |
| conditions | 01 | 02 | 07 | Control | Control | Control | type |
| EODFR | 1.70a | 2.5a | 2.5a | 2.8 | 3.9 | 3.4 | 3.9 |
| Control | 1.4 | 1.8 | 1.9 | 1.4 | 1.9 | 1.9 | 1.8 |

a= Statistically significantly different from wild type at p < 0.05, based on a two-tailed Student's t-test.

ME03531 events were also evaluated in the FRE assay. Under FRE conditions, the petiole length of seedlings from ME03531 events was statistically significantly shorter than the petiole length of seedlings of segregating progeny that did not contain the Ceres Clone 37493 (SEQ ID NO: 42) SAM dependent-carboxyl methyl transferase-like protein transgene.

### Example 9

### Analysis of ME01990, ME22238 and SR03598 Events

Constructs were generated according to the method described in Example 1. The cDNA Ceres Clone 258241 (SEQ ID NO: 40) was cloned into a Ti plasmid vector, CRS 338, which contains a phosphoinothricin acetylase transferase gene conferring Finale™ resistance on transformed plants, operably linked in the sense orientation relative to either a CaMV 35S constitutive promoter, a p326 promoter, or a PR0924 promoter. Wild-type Arabidopsis plants were transformed separately with each construct as described in Example 1.

Transgenic Arabidopsis lines containing Ceres Clone 258241 (SEQ ID NO: 40) operably linked to a CaMV 35S promoter, a p326 promoter (SEQ ID NO: 37) or a PR0924 promoter (SEQ ID NO: 66) were designated ME01990, ME22238, or SR03598, respectively. The presence of each vector containing a DNA clone described above in the respective transgenic Arabidopsis line transformed with the vector was confirmed by Finale™ resistance, PCR amplification from green leaf tissue extract, and/or sequencing of PCR products. As controls, wild-type Arabidopsis plants were transformed with the empty vector CRS 338. T1 seeds were germinated and allowed to self-pollinate. T2 seeds were collected and a portion was germinated, allowed to self-pollinate, and T3 seeds were collected.

The days to flowering, seed yield and dry weight at harvest were analyzed in homozygous T3 ME01990, ME22238, and T3 SR03598 plants cultured under in normal light growth conditions as follows. Seeds from transgenic T3 plants and corresponding control plants were sown in a checkerboard pattern in no-hole utility flats. Wild-type Arabidopsis thaliana ecotype Wassilewskija (Ws) plants and non-transgenic segregating plants were used as control plants. The flats were covered with propagation domes and maintained at 4° C. in the dark for three days. The flats were then transferred to a Conviron walk-in growth chamber (Controlled Environments Inc.) with a 16:8 hour light:dark cycle, a relative humidity of 70%, a temperature of 22° C., and an irradiance of about 150 μmol/m²/s of light having a red to far-red ratio greater than one. The propagation domes were removed after four days and the plants were cultivated under normal light growth conditions, i.e., a 16:8 hour light:dark cycle, a relative humidity of 70%, a temperature of 22° C., and an irradiance of about 150 μmol/m²/s of light having a red to far-red ratio greater than one, for the duration of the experiment. Six to nine replicate plant samples were measured at each stage for each treatment group, and the average values and standard deviations were calculated.

Dry weight and seed yield were determined after senescence, when the plants were eight weeks old. Seed and dry weight measurements were obtained when plants were eight weeks old. Plants were harvested individually and allowed to dry completely at 28° C. for three days. The seed was separated from the dried plant material using a sieve (300 μM mesh size) and weighed. The dried plant material was added to the seed and the combined weight was recorded as the dry weight.

Internal control plants for this experiment were T3 segregating progeny that did not contain the Ceres Clone 258241 (SEQ ID NO: 40) putative zinc-finger transgene. The T3 ME01990 analysis included event ME01990-2-20. The T3 ME22238 analysis included events ME22238-5-3 and ME22238-6-12. The T3 SR03598 analysis included events SR03598-1-2 and SR03598-2-5.

Under normal light growth conditions, days to flowering were increased in the ME01990-2-20, ME22238-5-3 and ME22238-6-12 events relative to days to flowering of the corresponding internal control plants; days to flowering for the SR03598 events were not observed to differ from the corresponding internal control plants. The seed yield of the ME1990-2-20 plants was increased relative to the seed yield in the corresponding internal control plants; no increases in seed yield were observed for any of the ME22238 or SR03598 events. The dry weight at harvest for the ME01990-2-20 and the ME22238-6-12 events was increased relative to that of the corresponding internal control plants; a moderate increase was also observed in the ME22238-5-3 event. The dry weight of the SR03598 events were not observed to differ from that of the corresponding internal control plants.

The response of T3 ME22238 or SR03598 plants to FRE conditions was analyzed in three different assays: the foliar canopy petiole length assay, the NL+Far-red enriched assay, and the End-of-day-Far-red (EODFR) assay as described in Examples 2, 3 and 4.

TABLE 5
FRE response in ME2238 and SR03598 Events

| | p326:ZF | | | | | | PR0924:ZF | | | | | |
|---|---|---|---|---|---|---|---|---|---|---|---|---|
| | Foliar Canopy petiole length (mm) | | | | | | Foliar Canopy petiole length (mm) | | | | | |
| | ME22238-5-4 | | | | | | SR03598-2-13 | | | | | |
| | Internal control | | ME22238-5-3 | | ME22238-6-12 | | Internal | | SR03598-1-2 | | SR03598-2-5 | |
| | | Foliar | | Foliar | | Foliar | control | | | Foliar | | Foliar |
| | NL | Canopy | NL | Canopy | NL | Canopy | NL | Foliar Canopy | NL | Canopy | NL | Canopy |
| average | 8.54 | 12.56 | 8.24 | 11.25 | 7.95 | 12.01 | 5.87 | 9.45 | 6.55 | 8.45 | 7 | 10.1 |
| St. Dev | 1.01 | 0.95 | 0.87 | 1.25 | 0.84 | 0.97 | 0.55 | 1.06 | 0.67 | 0.47 | 1.1 | 0.54 |
| p value | | | | NA | | NA | | | | NA | | NA |
| | p326:ZF | | | | | | PR0924:ZF | | | | | |
| | NL + FRE petiole length (mm) | | | | | | NL + FRE petiole length (mm) | | | | | |
| | ME22238-5-4 | | | | | | SR03598-2-13 | | | | | |
| | Internal control | | ME22238-5-3 | | ME22238-6-12 | | Internal control | | SR03598-1-2 | | SR03598-2-5 | |
| | NL | NL + FRE | NL | NL + FRE | NL | NL + FRE | NL | NL + FRE | NL | NL + FRE | NL | NL + FRE |
| average | 5.64 | 11.45 | 6.02 | 10.55 | 6.95 | 10.35 | 4.9 | 6.12 | 4.53 | 5.03 | 4.42 | 4.98 |
| St. Dev | 0.95 | 1.01 | 0.77 | 0.95 | 0.84 | 1.11 | 0.62 | 1.21 | 0.47 | 0.79 | 0.6 | 0.59 |
| p value | | | | NA | | NA | | | | 3.00E−02 | | 1.00E−02 |
| | p326:ZF | | | | | | | | | | | |
| | SD + EoDFR hypocotyl length (mm) | | | | | | PR0924:ZF | | | | | |
| | ME22238-5-4 | | | | | | SD + EoDFR hypocotyl length (mm) | | | | | |
| | Internal control | | | | | | SR03598-2-13 | | | | | |
| | | SD + | ME22238-5-3 | | ME22238-6-12 | | Internal control | | SR03598-1-2 | | SR03598-2-5 | |
| | SD | EoDFR | SD | SD + EoDFR | SD | SD + EoDFR | SD | SD + EoDFR | SD | SD + EoDFR | SD | SD + EoDFR |
| average | 1.96 | 3.17 | 1.48 | 2.69 | 1.59 | 2.73 | 1.67 | 3.12 | 1.42 | 2.69 | 1.53 | 2.64 |
| St. Dev | 0.86 | 0.47 | 0.22 | 0.21 | 0.22 | 0.28 | 0.07 | 0.13 | 0.01 | 0.16 | 0.01 | 0.12 |
| p value | | | | NA | | NA | | | | NA | | NA |

As shown in Table 5, a significant difference in petiole length was observed between T3 SR03598 events and the internal control in the NL+FRE petiole length assay. The internal control seedlings showed an average increase in petiole length of about 1.2 mm; the petiole length of the T3 SR03598 seedlings was statistically significantly shorter than that of the internal controls. No significant differences in petiole length or hypocotyl length were observed between T3 SR03598 events and the internal control in the foliar canopy petiole assay or the SD+EODFR hypocotyl length assay, respectively, although there was a trend toward shorter hypocotyl in the SD+EODRF assay.

No significant differences were observed between T3 ME22238 events and the internal control in the foliar canopy petiole assay, the NL+FRE petiole length assay or the SD+EODFR hypocotyl length assay.

### Example 10

### Analysis of Transgenic Rice Expressing Ceres Clone 258241

Constructs were generated according to the method described in Example 1. The cDNA Ceres Clone 258241 (SEQ ID NO: 40) was cloned into a Ti plasmid vector, CRS 338, which contains a phosphoinothricin acetylase transferase gene conferring Finale™ resistance on transformed plants, operably linked in the sense orientation relative to either a CaMV 35S constitutive promoter, a p326 (SEQ ID NO: 37) promoter, or a PR0924 (SEQ ID NO: 66) promoter. Each construct was introduced into a tissue culture of the rice cultivar Kitaake by an Agrobacterium-mediated transformation protocol according to the method described in “Efficient transformation of rice (Oryza sativa L.) mediated by Agrobacterium and sequence analysis of the boundaries of the T-DNA.” Hiei, Y., Ohta, S., Komari, T. and Kumashiro, T. Plant J. 6, 271-282 (1994).

Plant height and days to flowering were analyzed in homozygous T2 Ceres Clone 258241 plants cultured under normal light growth conditions of 16 hr light, 8 hr dark cycle at 28 C, 600-800 μmol/meter² per second. About 8-10 replicates were used per event. Control plants were non-transgenic segegrants. Plant height was measured in the p326 promoter containing plants p326-c258241-37 and p326-c258241-5 and the PR0924 promoter containing plants PR0924-c258241-2 and PR0924-c258241-5. Days to flowering were measured in the p326-c258241-37 and the PR0924-c258241-5 plants.

As shown in Table 6, under normal light growth conditions, the height of the p326-c258241-37, p326-c258241-5 and the PR0924-c258241-5 plants was significantly less than that of the control plants. The p326-c258241-37, p326-c258241-5 and the PR0924-c258241-5 plants were, on average 27, 12.6, and 13.75 mm shorter than the control plants. No significant differences were observed between the height of the PR0924-c258241-2 and the control plants. The number of days to flowering for the p326-c258241-37 and the PR0924-c258241-5 plants was significantly greater than that of the control plants. The p326-c258241-37 and the PR0924-c258241-5 plants flowered, on average 7 and 8 days later, respectively, than did the control plants.

TABLE 6
Plant height and days to flowering in transgenic rice expressing
Ceres Clone 258241

| | 326F:ZF | | | | | |
|---|---|---|---|---|---|---|
| | Adult Plant Height | | | PR0924:ZF | | |
| | p326- | | | Adult Plant Height | | |
| | c258241- | p326- | con- | PR0924- | PR0924- | con- |
| | 37 | c258241-5 | trol | c258241-2 | c258241-5 | trol |
| average | 51.67a | 66.17a | 78.75 | 72.6 | 65a | 78.75 |
| St. Dev | 3.31 | 6.94 | 8.63 | 13.13 | 6.2 | 8.63 |
| p value | 3.89E−03 | 3.37E−02 | | 4.48E−01 | 1.82E−02 | |

a= Statistically significantly different from control at p < 0.05, based on a two-tailed Student's t-test.

### Example 11

### Analysis of ME03531, ME22242 and SR03597 Events

Constructs were generated according to the method described in Example 1. The cDNA Ceres Clone 37493 (SEQ ID NO: 42) was cloned into a Ti plasmid vector, CRS 338, which contains a phosphoinothricin acetylase transferase gene conferring Finale™ resistance on transformed plants, operably linked in the sense orientation relative to either a CaMV 35S constitutive promoter, a p326 promoter, or a PR0924 promoter. Wild-type Arabidopsis plants were transformed separately with each construct. The transformations were performed essentially as described in Bechtold et al., C.R. Acad. Sci. Paris, 316:1194-1199 (1993).

Transgenic Arabidopsis lines containing Ceres Clone 37493 (SEQ ID NO: 42) operably linked to a CaMV 35S promoter, a p326 promoter or a PR0924 promoter were designated ME03531, ME22242, or SR03597, respectively. The presence of each vector containing a DNA clone described above in the respective transgenic Arabidopsis line transformed with the vector was confirmed by Finale™ resistance, PCR amplification from green leaf tissue extract, and/or sequencing of PCR products. As controls, wild-type Arabidopsis plants were transformed with the empty vector CRS 338. T1 seeds were germinated and allowed to self-pollinate. T2 seeds were collected and a portion was germinated, allowed to self-pollinate, and T3 seeds were collected.

The days to flowering, seed yield and dry weight at harvest were analyzed in homozygous T3 ME03531, ME22242, or SR03597 plants cultured under in normal light growth conditions as described in Example 8. Internal control plants for this experiment were T3 segregating progeny that did not contain the Ceres Clone 37493 (SEQ ID NO: 42) SAM-dependent carboxyl methyl transferase transgene. The T3 ME03531 analysis included event ME03531-7-11; segregating progeny of ME03531-7-9 were used as an Internal Control. The T3 ME22242 analysis included events ME22242-6-1 and ME22242-2-6; segregating progeny of ME22242-6-11 were used as an Internal Control. The T3 SR03597 analysis included events SR03597-2-5 and SR03597-3-2; segregating progeny of SR03597-3-13 were used as an Internal Control.

Under normal light growth conditions, days to flowering were significantly increased in the T3 ME03531 and ME22242 events relative to the corresponding internal controls. Days to flowering for the T3 SR03597 events were not observed to differ from the corresponding internal controls. Seed yields under normal light conditions were significantly decreased in the T3 ME03531 and ME22242 events relative to the seed yields of the internal controls; the seed yields for the T3 SR03597 events were not observed to differ significantly from those of the corresponding internal controls. Dry weight at harvest under normal light conditions were significantly decreased in the T3 ME03531 and ME22242 events relative to the dry weight at harvest of the internal controls; the dry weight at harvest for the T3 SR03597 events was not significantly different from internal controls.

The response of T3 ME22242 or SR03597 plants to FRE conditions was analyzed in three different assays: the foliar canopy petiole length assay, the NL+Far-red enriched assay, and the End-of-day-Far-red (EODFR) assay as described in Examples 2, 3 and 4. The T3 ME22242 analysis included events ME22242-6-1 and ME22242-2-6; segregating progeny of ME22242-6-11 were used as an Internal Control. The T3 SR03597 analysis included events SR03597-2-5 and SR03597-3-2; segregating progeny of SR03597-3-13 were used as an Internal Control.

As indicated in Table 7, the FRE responses of the seedlings expressing the Ceres Clone 37493 (SEQ ID NO: 42) SAM-dependent carboxyl methyl transferase differed from those of the corresponding segregating progeny that did not express the transgene. The petiole length of seedlings of the T3 ME22242 events was statistically significantly shorter than those of the internal control in both the foliar canopy petiole length assay and the NL+FRE petiole length assay. Under FRE conditions in the EODFR hypocotyl length assay, the hypocotyl length of seedlings of the T3 ME22242 events was statistically significantly shorter than those of the internal control. Under FRE conditions in both the foliar canopy petiole length assay and the NL+FRE petiole length assay, the petiole length of seedlings of the T3 SR03597 events was statistically significantly shorter than those of the internal control. Under FRE conditions in the EODFR hypocotyl length assay, the hypocotyl length of seedlings of the T3 SR03597 events was statistically significantly shorter than those of the internal control.

TABLE 7
FRE response in ME2242 and SR03597 Events

| | p326:SAM | | | | | | PR0924:SAM | | | | | |
|---|---|---|---|---|---|---|---|---|---|---|---|---|
| | Foliar Canopy petiole length (mm) | | | | | | Foliar Canopy petiole length (mm) | | | | | |
| | ME22242-6-11 | | | | | | SR03597-03-13 | | | | | |
| | Internal Control | | ME22242-2-6 | | ME22242-6-1 | | Internal Control | | SR03597-02-05 | | SR03597-03-02 | |
| | | Foliar | | Foliar | | Foliar | | Foliar | | Foliar | | Foliar |
| | NL | Canopy | NL | Canopy | NL | Canopy | NL | Canopy | NL | Canopy | NL | Canopy |
| average | 2.01 | 3.28 | 2.25 | 2.7a | 2.3 | 2.58a | 1.75 | 2.6 | 2.05 | 2.15a | 1.57 | 1.67a |
| St. Dev | 0.27 | 0.66 | 0.38 | 0.4 | 0.5 | 0.53 | 0.27 | 0.52 | 0.48 | 0.34 | 0.33 | 0.49 |
| p value | | | | 2.30E−03 | | 5.10E−04 | | | | 4.10E−03 | | 2.30E−04 |
| | p326:SAM | | | | | | PR0924:SAM | | | | | |
| | NL + FRE petiole length (mm) | | | | | | NL + FRE petiole length (mm) | | | | | |
| | ME22242-6-11 | | | | | | SR03597-03-13 | | | | | |
| | Internal Control | | ME22242-2-6 | | ME22242-6-1 | | Internal Control | | SR03597-02-05 | | SR03597-03-02 | |
| | NL | NL + FRE | NL | NL + FRE | NL | NL + FRE | NL | NL + FRE | NL | NL + FRE | NL | NL + FRE |
| average | 1.58 | 5.58 | | 2.72a | | 3.51a | 4.49 | 5.68 | 4.34 | 2.95a | 3.25 | 5.02a |
| St. Dev | 0.29 | 0.95 | | 0.43 | | 0.82 | 0.85 | 0.78 | 0.55 | 0.74 | 0.62 | 0.62 |
| p value | | | | 0.02 | | 3.00E−06 | | | | 1.07E−05 | | 4.00E−02 |
| | p326:SAM | | | | | | PR0924:SAM | | | | | |
| | SD + EoDFR hypocotyl length (mm) | | | | | | SD + EoDFR hypocotyl length (mm) | | | | | |
| | ME22242-6-11 | | | | | | SR03597-03-13 | | | | | |
| | Internal Control | | ME22242-2-6 | | ME22242-6-1 | | Internal Control | | SR03597-02-05 | | SR03597-03-02 | |
| | | SD + | | SD + | | SD + | | SD + | | SD + | | SD + |
| | SD | EoDFR | SD | EoDFR | SD | EoDFR | SD | EoDFR | SD | EoDFR | SD | EoDFR |
| average | 1.58 | 3.23 | 1.6 | 2.12a | 1.68 | 2.56a | 1.49 | 3.65 | 1.56 | 2.41a | 1.47 | 2.57a |
| St. Dev | 0.29 | 0.43 | 0.28 | 0.24 | 0.22 | 0.31 | 0.01 | 0.33 | 0.03 | 0.12 | 0.05 | 0.12 |
| p value | | | | 2.96E−07 | | 4.18E−04 | | | | 2.50E−04 | | 5.00E−03 |

a= Statistically significantly different from control at p < 0.05, based on a two-tailed Student's t-test.

### Example 12

### Analysis of Transgenic Rice Expressing Ceres Clone 37493

Constructs were generated according to the method described in Example 1. The cDNA Ceres Clone 37493 (SEQ ID NO: 42) was cloned into a Ti plasmid vector, CRS 338, which contains a phosphinothricin acetyl transferase gene conferring Finale™ resistance on transformed plants, operably linked in the sense orientation relative to either a CaMV 35S constitutive promoter, a p326 promoter, or a PR0924 promoter. Each construct was introduced into a tissue culture of the rice cultivar Kitaake by an Agrobacterium-mediated transformation protocol.

Plant height and days to flowering were analyzed in homozygous T2 Ceres Clone 37493 plants cultured under normal light growth conditions of 16 hr light, 8 hr dark cycle at 28 C, 600-800 μmol/meter²/second. About 8-10 replicates were used per event. Control plants were non-transgenic segegrants or wild type rice plants. Plant height was measured in the p326 promoter containing plants p326-c37493-2 and p326-c37493-16 and the PR0924 promoter containing plants PR0924-c37493-F and PR0924-c37493-5. Days to flowering were measured in the p326-c37493-2 and the p326-c37493-16 plants.

The result of this experiment are shown in Table 8. Under normal light growth conditions, the height of the p326-c37493-2, p326-c37493-16 and the PR0924-c37493-5 plants was significantly less than that of the wild type plants. The p326-c37493-2, p326-c37493-16 and the PR0924-c37493-5 plants were, on average 25.5, 12.75, and 18.55 mm shorter than the wild type plants. No significant differences were observed between the height of the PR0924-c37493-F and the wild type plants.

Under normal light growth conditions, the number of days to flowering of the p326-c37493-2 and the p326-c37493-16 plants was significantly greater than that of the wild type plants. The p326-c37493-2 and the p326-c37493-16 plants flowered, on average 9.75 and 8.75 days later, respectively, than did the wild type plants.

TABLE 8
Plant height and days to flowering in transgenic rice expressing
Ceres Clone 37493

| | 326F:SAM | | | PR0924:SAM | | |
|---|---|---|---|---|---|---|
| | Adult Plant Height | | | Adult Plant Height | | |
| | p326- | p326- | wild | PR0924- | p326- | wild |
| | c37493-2 | c37493-16 | type | c37493-F | c37493-5 | type |
| average | 51a | 63.75a | 76.5 | 71.67 | 60.2a | 78.75 |
| St. Dev | 3.79 | 3.3 | 6.49 | 8.96 | 5.22 | 8.63 |
| p value | 4.65E−05 | 1.28E−02 | | 2.49E−01 | 5.09E−03 | |

a= Statistically significantly different from control at p < 0.05, based on a two-tailed Student's t-test.

### Example 13

### Determination of Functional Homolog and/or Orthologue Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO: 43 are shown in FIG. 1. The percent identities of functional homologs and/or orthologs to SEQ ID NO: 82 are shown in Table 11. An HMM was generated using the following sequences as input: SEQ ID NOs:43 and 45-58. The sequences are aligned in FIG. 1. When fitted to the HMM, the sequences had the HMM bit scores listed in Table 11.

TABLE 11
Percent identity to Ceres Clone 37493

| | | SEQ ID | | | HMM |
|---|---|---|---|---|---|
| Designation | Species | NO: | % identity | e-value | score |
| Ceres Clone ID | Arabidopsis | 43 | N/A | N/A | 814.3 |
| no. 37493 | thaliana | | | | |
| Ceres gDNA ID | Populus | 45 | 76.5 | 3.00E−156 | 821.1 |
| no. 1494370 | balsamifera | | | | |
| | subsp. | | | | |
| | trichocarpa | | | | |
| Public GI no. | Oryza sativa | 46 | 65.5 | 8.49E−127 | 791 |
| 50929439 | subsp. japonica | | | | |
| Public GI no. | Cucumis sativus | 47 | 40.4 | 2.19E−48 | 847 |
| 18461100 | | | | | |
| Public GI no. | Arabidopsis | 48 | 38 | 1.39E−46 | 813.5 |
| 37904506 | lyrata subsp. | | | | |
| | lyrata | | | | |
| Public GI no. | Cestrum | 49 | 37.9 | 8.59E−47 | 797.5 |
| 58201418 | nocturnum | | | | |
| Public GI no. | Brassica rapa | 50 | 37.3 | 1.09E−44 | 877.8 |
| 6651395 | subsp. | | | | |
| | pekinensis | | | | |
| Public GI no. | Brassica juncea | 51 | 37.3 | 1.09E−44 | 877.7 |
| 55442027 | | | | | |
| Public GI no. | Capsicum | 52 | 37.3 | 1.79E−44 | 855.6 |
| 77745528 | annuum | | | | |
| Public GI no. | Petunia x | 53 | 37.2 | 5.09E−49 | 892.7 |
| 28629495 | hybrida | | | | |
| Public GI no. | Nicotiana | 54 | 37.1 | 3.60E−48 | 903.9 |
| 58201458 | tabacum | | | | |
| Public GI no. | Antirrhinum | 55 | 36.9 | 4.09E−47 | 822.6 |
| 9789277 | majus | | | | |
| Public GI no. | Medicago | 56 | 36.8 | 2.09E−45 | 814.5 |
| 87241303 | truncatula | | | | |
| Public GI no. | Clarkia breweri | 57 | 36.2 | 8.20E−42 | 851.7 |
| 6002712 | | | | | |
| Public GI no. | Camellia | 58 | 36.1 | 2.80E−48 | 800.2 |
| 59611829 | sinensis | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A plant comprising an exogenous nucleic acid, said exogenous nucleic acid comprising a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, wherein said plant has a statistically significant difference in a response to far-red-enriched light conditions as compared to the corresponding response in a control plant that does not comprise said regulatory region operably linked to said nucleotide sequence.

2. The plant of claim 1, wherein said sequence identity is 85 percent or greater.

3. The plant of claim 1, wherein said sequence identity is 90 percent or greater.

4. The plant of claim 1, wherein said sequence identity is 95 percent or greater.

5. A plant comprising (a) a first exogenous nucleic acid comprising a nucleotide sequence encoding a polypeptide having 80 percent or greater sequence identity to an amino acid sequence selected from the group consisting of SEQ ID NO: 41 operably linked to a regulatory region and (b) a second exogenous nucleic acid comprising a nucleotide sequence encoding a polypeptide having 80 percent or greater sequence identity to an amino acid sequence selected from the group consisting of SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 operably linked to a regulatory region; wherein said plant has a statistically significant difference in a response to far-red-enriched light conditions as compared to the corresponding response in a control plant that does not comprise said first and said second exogenous nucleic acids.

6. A plant comprising an exogenous nucleic acid, said exogenous nucleic acid comprising a regulatory region operably linked to a nucleotide sequence encoding a polypeptide wherein the HMM bit score of the amino acid sequence of said polypeptide is greater than 395, said HMM based on the amino acid sequences depicted in FIG. 1, and wherein said plant has a statistically significant difference in a response to far-red-enriched light conditions as compared to the corresponding response in a control plant that does not comprise said exogenous nucleic acid.

7. A plant of claim 6, wherein the HMM bit score is 790 or greater.

8. The plant of claim 1, wherein said nucleotide sequence encodes a polypeptide comprising an amino acid sequence corresponding to SEQ ID NO: 41.

9. The plant of claim 1, wherein said nucleotide sequence encodes a polypeptide comprising an amino acid sequence corresponding to SEQ ID NO: 43.

10. The plant of claim 1, wherein said regulatory region is a promoter.

11. The plant of claim 10, wherein said promoter is a tissue-preferential, broadly expressing, or inducible promoter.

12. The plant of claim 1, wherein said plant is a dicot.

13. The plant of claim 12, wherein said plant is a member of the genus Brassica spp., Brassica napus, Brassica rapa, Brassica oleracea, Glycine max, Gossypium spp., Gossypium hirsutum, Gossypium herbaceum, Helianthus annuus, Lactuca sativa, Medicago saliva.

14. The plant of claim 1, wherein said plant is a monocot.

15. The plant of claim 14, wherein said plant is a member of the genus Avena saliva, Hordeum vulgare, Oryza sativa, Panicum virgatum, Secale cereale, Triticum aestivum, and Zea mays.

16. The plant of claim 1, wherein said difference in response to FRE conditions is a difference in petiole length.

17. The plant of claim 1, wherein said difference in response to FRE conditions is a difference in hypocotyl length.

18. The plant of claim 1, wherein said far-red-enriched light conditions comprise a red:far-red (R:FR) ratio of less than 1.0.

19. The plant of claim 18, wherein said R:FR ratio is from about 0.05 to about 0.9.

20. The plant of claim 18, wherein said R:FR ratio is from about 0.10 to about 0.7.

21. The plant of claim 18, wherein said R:FR ratio is from about 0.10 to about 0.5.

22. The plant of claim 18, wherein said R:FR ratio is from about 0.10 to about 0.3.

23. The plant of claim 18, wherein said R:FR ratio is about 0.22.

24. The plant of claim 18, wherein said R:FR ratio is about 0.14.

25. The plant of claim 1, wherein said far-red-enriched light conditions comprise continuous FRE conditions.

26. The plant of claim 1, wherein said far-red-enriched light conditions comprise a pulse of FRE conditions.

27. The plant of claim 26, wherein said pulse of FRE conditions comprises about 0.1 to about 8.0 hours of FRE conditions per day.

28. The plant of claim 26, wherein said pulse of FRE conditions comprises about 0.2 to about 6.0 hours of FRE conditions per day.

29. The plant of claim 27, wherein said pulse of FRE conditions comprises about 0.3 to about 3.0 hours of FRE conditions per day.

30. The plant of claim 27, wherein said pulse of FRE conditions comprises about 0.4 to about 2.0 hours of FRE conditions per day.

31. The plant of claim 27, wherein said pulse of FRE conditions comprises about 0.5 hours of FRE conditions per day.

32. The plant of claim 31, wherein said 0.5 hour pulse of FRE conditions occurs at the end of the day.

33. Progeny of the plant of claim 1, wherein said progeny has a statistically significant difference in a response to far-red-enriched light conditions as compared to the corresponding response in a control plant that does not comprise said regulatory region operably linked to a nucleotide sequence.

34. A method of producing a crop, said method comprising: growing a plurality of plants comprising an exogenous nucleic acid comprising a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, wherein said plant has a statistically significant difference in a response to far-red enriched light conditions as compared to the corresponding response in a control plant that does not comprise said regulatory region operably linked to a nucleotide sequence; and harvesting said crop from said plants

35. A method of producing a crop, said method comprising: growing a plurality of plants comprising an exogenous nucleic acid comprising a regulatory region operably linked to a nucleotide sequence encoding a polypeptide wherein the HMM bit score of the amino acid sequence of said polypeptide is greater than 790, said HMM based on the amino acids sequences depicted in FIG. 1, and wherein said plant has a statistically significant difference in a response to far-red-enriched light conditions as compared to the corresponding response in a control plant that does not comprise said regulatory region operably linked to said nucleotide sequence; and harvesting said crop from said plants.

36. Seed from a transgenic plant according to claim 1.

37. Vegetative tissue from a transgenic plant according to claim 1.

38. Fruit from a transgenic plant according to claim 1.

39. An article of manufacture comprising:
a) packaging material; and
b) seeds within said packaging material, said seeds comprising an exogenous nucleic acid, said exogenous nucleic acid comprising a regulatory region operably linked to a nucleotide sequence encoding a polypeptide having 80 percent or greater sequence identity to an amino acid sequence selected from the group consisting of SEQ ID NO: 41, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, wherein plants grown from said seeds have a statistically significant difference in a response to far-red-enriched light conditions as compared to the corresponding response in a control plant that does not comprise said regulatory region operably linked to said nucleotide sequence.

40. An article of manufacture comprising:
a) packaging material; and
b) seeds within said packaging material, said seeds comprising an exogenous nucleic acid, said exogenous nucleic acid comprising a regulatory region operably linked to a nucleotide sequence encoding a polypeptide wherein the HMM bit score of the amino acid sequence of said polypeptide is greater than 790, said HMM based on the amino acids sequences depicted in FIG. 1, and wherein said plant has a statistically significant difference in a response to far-red-enriched light conditions as compared to the corresponding response in a control plant that does not comprise said regulatory region operably linked to said nucleotide sequence

41. The article of claim 39, said regulatory region comprising a first transcription activator recognition site and a first promoter, said seeds further comprising:
i) a second exogenous nucleic acid, said second exogenous nucleic comprising a second transcription activator recognition site and a second promoter, said second recognition site and said second promoter operably linked to a sequence causing seed infertility; and
ii) at least one activator nucleic acid encoding at least one transcription activator that binds to at least one of said recognition sites, each said at least one transcription activator having a promoter operably linked thereto, wherein plants grown from said seeds are infertile


SEQUENCE LISTING (68 sequences)

SEQ ID NO: 1 - DNA, 1823 bp, Arabidopsis thaliana
  misc_feature: Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature: Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature: Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature: Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1022 bp, Arabidopsis thaliana
  misc_feature: Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 6 - DNA, 1000 bp, Arabidopsis thaliana
  misc_feature: Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 7 - DNA, 998 bp, Arabidopsis thaliana
  misc_feature: Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 8 - DNA, 1000 bp, Arabidopsis thaliana
  misc_feature: Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 9 - DNA, 1000 bp, Arabidopsis thaliana
  misc_feature: Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 10 - DNA, 1024 bp, Arabidopsis thaliana
  misc_feature: Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 11 - DNA, 1000 bp, Arabidopsis thaliana
  misc_feature: Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 12 - DNA, 921 bp, Arabidopsis thaliana
  misc_feature: Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 13 - DNA, 669 bp, Arabidopsis thaliana
  misc_feature: Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 14 - DNA, 702 bp, Arabidopsis thaliana
  misc_feature: Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 15 - DNA, 397 bp, Arabidopsis thaliana
  misc_feature: Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 16 - DNA, 1024 bp, Arabidopsis thaliana
  misc_feature: Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 17 - DNA, 999 bp, Arabidopsis thaliana
  misc_feature: Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 18 - DNA, 1020 bp, Arabidopsis thaliana
  misc_feature: Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 19 - DNA, 1000 bp, Arabidopsis thaliana
  misc_feature: Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 20 - DNA, 1003 bp, Arabidopsis thaliana
  misc_feature: Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 21 - DNA, 1000 bp, Arabidopsis thaliana
  misc_feature: Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 22 - DNA, 1005 bp, Arabidopsis thaliana
  misc_feature: Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 23 - DNA, 1002 bp, Arabidopsis thaliana
  misc_feature: Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 24 - DNA, 1024 bp, Arabidopsis thaliana
  misc_feature: Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 25 - DNA, 911 bp, Arabidopsis thaliana
  misc_feature: Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 26 - DNA, 996 bp, Arabidopsis thaliana
  misc_feature: Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 27 - DNA, 1000 bp, Arabidopsis thaliana
  misc_feature: Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 28 - DNA, 1000 bp, Arabidopsis thaliana
  misc_feature: Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 29 - DNA, 1000 bp, Arabidopsis thaliana
  misc_feature: Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 30 - DNA, 998 bp, Arabidopsis thaliana
  misc_feature: Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 31 - DNA, 999 bp, Arabidopsis thaliana
  misc_feature: Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 32 - DNA, 1000 bp, Arabidopsis thaliana
  misc_feature: Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 33 - DNA, 999 bp, Arabidopsis thaliana
  misc_feature: Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 34 - DNA, 998 bp, Arabidopsis thaliana
  misc_feature: Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 35 - DNA, 1000 bp, Arabidopsis thaliana
  misc_feature: Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 36 - DNA, 1514 bp, Arabidopsis thaliana
  misc_feature: Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 37 - DNA, 1954 bp, Arabidopsis thaliana
  misc_feature: Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 38 - DNA, 2016 bp, Arabidopsis thaliana
  misc_feature: Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 39 - DNA, 667 bp, Arabidopsis thaliana
  misc_feature: Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 40 - DNA, 588 bp, Arabidopsis thaliana
  misc_feature: Ceres CLONE ID no. 258241
aaatcttctttcctcaatatcacccagaagaaagagaaagtgaaacagatcaagaaagagattgagaaaaatgtgtagaggcttgaataatgaagagagcagaagaagtgacggaggaggttgccggagtctctgcacgagaccgagtgttccggtaaggtgtgagctttgcgacggagacgcctccgtgttctgtgaagcggactcggcgttcctctgtagaaaatgtgaccggtgggttcatggagcgaattttctagcttggagacacgtaaggcgcgtgctatgcacttcttgtcagaaactcacgcgccggtgcctcgtcggagatcatgacttccacgttgttttaccgtcggtgacgacggtcggagaaaccaccgtggagaatagaagtgaacaagataatcatgaggttccgtttgtttttctctgattattagatttttttggtgtaagctataaatattctagggtttttatgcaattttcttttaagaaaaatctgaaaattttaattttatgaagcttagaattttaagaaatgtgtaatgaaaaaaaaaaaagaattttaagaaatgggtagtg

SEQ ID NO: 41 - protein, 121 aa, Arabidopsis thaliana
  misc_feature: Ceres CLONE ID no. 258241
MCRGLNNEESRRSDGGGCRSLCTRPSVPVRCELCDGDASVFCEADSAFLCRKCDRWVHGANFLAWRHVRRVLCTSCQKLTRRCLVGDHDFHVVLPSVTTVGETTVENRSEQDNHEVPFVFL

SEQ ID NO: 42 - DNA, 1211 bp, Arabidopsis thaliana
  misc_feature: Ceres CLONE ID no. 37493
aaaaagagaatgggttctaagggagacaacgttgctgtatgtaacatgaagctcgagagacttctcagcatgaaaggtggcaaaggacaagacagctacgccaataactctcaagctcaggcaatgcatgcacggtccatgcttcacctcctagaggaaacacttgaaaatgttcacctcaactcctccgccagtccgccaccgttcacgacggttgatcttggctgttcctccggcgcaaatactgtccacataatcgatttcatagtcaaacatatctctaagaggtttgatgctgccggaatcgaccctccggagttcacagctttcttctccgatctcccaagcaacgacttcaacacgcttttccagcttcttccaccacttgtctctaacacttgcatggaggagtgtcttgctgccgacgggaaccgctcctacttcgtcgctggtgttcccggatcgttttaccggcgactttttccagcgagaaccattgactttttccactctgccttctccttgcattggctctctcaggtgccggaaagtgtgacggataggagatcggcggcgtacaatagagggagagttttcatacacggtgccggagagaagacaacaacggcatacaaacggcagtttcaggccgacttagcggagtttttgagggcaagagcggcagaggttaagagaggtggggccatgttcctggtctgtcttggtcgtacctctgtggatcccaccgaccagggtggtgccggtctactcttcggcactcattttcaggacgcttgggacgaccttgtccgcgagggtttggtagcagcagagaaacgagatggattcaatatcccggtgtacgcaccaagcttgcaagatttcaaggaagtagttgacgccaacggctcatttgcaattgataagctggtagtatacaaaggagggagtccacttgtggtgaatgagccagacgatgcatcagaagttggtcgtgcctttgctagtagttgtcggagtgtggccggagtgctcgtggaagctcacataggcgaagagctaagcaataaactgttctcacgagttgaaagccgagccactagccatgccaaagatgtcttggtgaatttgcagttcttccacattgtcgcttctttgtcttttacttaattagtaccaaataatatgaaaaagaaatatgttcgtgtgcc

SEQ ID NO: 43 - protein, 386 aa, Arabidopsis thaliana
  misc_feature: Ceres CLONE ID no. 37493
MGSKGDNVAVCNMKLERLLSMKGGKGQDSYANNSQAQAMHARSMLHLLEETLENVHLNSSASPPPFTTVDLGCSSGANTVHIIDFIVKHISKRFDAAGIDPPEFTAFFSDLPSNDFNTLFQLLPPLVSNTCMEECLAADGNRSYFVAGVPGSFYRRLFPARTIDFFHSAFSLHWLSQVPESVTDRRSAAYNRGRVFIHGAGEKTTTAYKRQFQADLAEFLRARAAEVKRGGAMFLVCLGRTSVDPTDQGGAGLLFGTHFQDAWDDLVREGLVAAEKRDGFNIPVYAPSLQDFKEVVDANGSFAIDKLVVYKGGSPLVVNEPDDASEVGRAFASSCRSVAGVLVEAHIGEELSNKLFSRVESRATSHAKDVLVNLQFFHIVASLSFT

SEQ ID NO: 44 - DNA, 1158 bp, Populus balsamifera subsp. trichocarpa
  misc_feature: Ceres ANNOT ID no. 1494370
atggctcctaaaggtgacaatgttgttgtgtctagtatgaagcttgagaagttgctttgcatgaagggaggcaaaggagaggcaagctatgccaataactctcaagctcaggccttacatgctcgatccatgcttcaccttctggaagaaaccctagatagggtgcacctaaactccccagaattccctttccaggtggcggacttagggtgctcatctggcaacaacaccatccacatcattgatgtcatcatcaagcatatgatcaagcgattcgagtcatccggactcgagccgccggagttctctgcttttttcgccgatctccctagcaatgacttcaacacccttttccagctcctccctcccccggccaattatgggggtagtatggaagagtgcctagctgctagtggccatcgaaactattttgcagctggagttcccggctcctttcaccggaggctttttccggcaagatcaattgacgttttccactcggcattttccttgcactggctctctcaggtgccggagtgtgttctagataagagatcagcagcatataacaaggggagggtgtttatccacaatgcaagcgagtccacaacaaatgcatacaagaaacagttccaaacagacctcgccggcttcctaagtgcaagatctcaagaaatgaagagcggtgggtccatgtttcttgtctgcttgggcagaacttctgccgaccccacagaccaaggtggggctggcctcctcttcgggacccactttcaggatgcctgggatgatcttgtccaggagggtctgattactagcgagaagcgtgacaatttcaacattccagtgtatgcaccaagcctccaagacttcaaggaagtagtggaagccaatggctcatttaccatagacaagctcgaggttttcaaaggagggagccctctggtggttaaccatcctgacaacgaagctgaggtcagccgggccatggctaattcctgccggagtgtggccggggtccttgttgatgcccacattggtgatgggctaagcgaggagttgttcttacgggtggagcaccgagccaaaagccatgcgaaagagcttctagagaagctgcaattcttccatatagttgcatctctttcttttgcttga

SEQ ID NO: 45 - protein, 385 aa, Populus balsamifera subsp. trichocarpa
  misc_feature: Ceres ANNOT ID no. 1494370
MAPKGDNVVVSSMKLEKLLCMKGGKGEASYANNSQAQALHARSMLHLLEETLDRVHLNSPEFPFQVADLGCSSGNNTIHIIDVIIKHMIKRFESSGLEPPEFSAFFADLPSNDFNTLFQLLPPPANYGGSMEECLAASGHRNYFAAGVPGSFHRRLFPARSIDVFHSAFSLHWLSQVPECVLDKRSAAYNKGRVFIHNASESTTNAYKKQFQTDLAGFLSARSQEMKSGGSMFLVCLGRTSADPTDQGGAGLLFGTHFQDAWDDLVQEGLITSEKRDNFNIPVYAPSLQDFKEVVEANGSFTIDKLEVFKGGSPLVVNHPDNEAEVSRAMANSCRSVAGVLVDAHIGDGLSEELFLRVEHRAKSHAKELLEKLQFFHIVASLSFA

SEQ ID NO: 46 - protein, 400 aa, Oryza sativa subsp. japonica
  misc_feature: Public GI no. 50929439
MASMKGENVTVSAAAAPRMKKLASMLCMKGGNGDGSYLNNSQAQALHARRMLHFLEETLDAMMERSSSDKLFTAADLGCSCGSNSLFIVDVIVRRVSEAYESRGRDAPEFQVFFSDLPSNDFNTLFQLLPPLLAPVAGSLEECLAAGEGAATATRPYHAAGVPGTFYGRLFPGESIDVFTSTFSLHWLSQVPEEVGDSASPAYNGGRVFVHRATEAVAAAYKRQFQADLARFLRSRAREMKRGGAMFLACLGRSSGDPADQGGAGLLFGTHFQDAWDDLVQEGVVEGEKRDSFNIPVYAPSLQEFRDVVRADGAFAIDRLELVRGGSPLVVDRPDDAAEVGRAMANSCKAVAGVLVDAHIGERRGAQLFERLERRAARHARELVEKMHFFHVVCSLSLAP

SEQ ID NO: 47 - protein, 370 aa, Cucumis sativus
  misc_feature: Public GI no. 18461100
MLHVNGGMGNTSYANNSRLQREIISMTCSIAKEALTNFYNQHIPTSITIADLGCSSGQNTLMLVSYLIKQVEEIRQKLHQRLPLEYQIFLNDLHGNDFNAVFTSLPRFLEDLGTQIGGDFGPCFFNGVPGSFYARLFPTKSVHFFHSSSSLHWLSRVPVGIENNKGNIYIGSTSPKSVGEAYYKQFQKDFSMFLKCRAEELVMGGGMVLTLVGRTSEDPSKSGGYYIWELLGLALNTMVAEGIVEEKKADSFNIPYYIPSPKEVEAEVVKEGSFILNQLKASSINLNHTVHKTEEESSTPLINNSLADATDYDFAKCIQSVSEPLLIRHFGEAIMDELFIRHRNIVAGCMAKHRIMECINLTISLTKKIS

SEQ ID NO: 48 - protein, 380 aa, Arabidopsis lyrata subsp. lyrata
  misc_feature: Public GI no. 37904506
MDPRFINTIPSLSYNDDKCDDEYAFVKALCMSGGDGTNSYSANSRLQRKVLTMAKPVLVKTTEEMMMSLDFPTYIKVAELGCSSGQNTFLAISEIINTISVLCQHVNKNPPEIDCCLNDLPENDFNTTFKFVPFFNKELMITSKASCFVYGAPGSFYSRLFSRNSLHIIHSSYALHWLSKVPEKLENNKGNVYITSSSPQSAYKAYLNQFQKDFSMFLRLRSEEIVSNGRMVLTFIGRNTLNDPLYRDCCHFWTLLSKSLRDLVFEGLVSESKLDAFNMPFYDPNVQELKQVIRNEGSFEINELETHGFDLGHSNYEEDDYEAGHDEANCIRAVSEPMLVAHFGEDIIDTLFDKYAHHVTQHANCRNKTTVSLVVSLTKK

SEQ ID NO: 49 - protein, 340 aa, Cestrum nocturnum
  misc_feature: Public GI no. 58201418
MNGGIGDISYANNSLVQQKVILMTKPITEQAITDLYNSLIFPQTLHIADLGCSSGANTFLVISEFVKIIEKQXKIHGFESPEFNFNFNDLPGNDFNTIFRSLGAFEEDLRMQVGENLGPCFFKGVPGSFYXRLFPSKSLHFVHSSYSLMWLSQVPEMTETNKXNIYMASTSPPSVIKAYYKQYESDFTSFLKYRSEELMKGGKMVLTFLGRESEDACSKECCYIWELLAKALNELVQEGLXEEXKLDSFNIPQYTPSPAEVKYIVGKHGSFAVNRLESSRVHWNVTNNNNNSINGGYNVSRCMRAVAEPLLVSHFGEDLMDLVFQKYEGIISECMAKEKT

SEQ ID NO: 50 - protein, 392 aa, Brassica rapa subsp. pekinensis
  misc_feature: Public GI no. 6651395
MEVMRILHMNKGNGETSYAKNSIVQSNIISLGRRVMDEALKKLMIRNSEILSFGIADLGCSSGPNSLLSISNIVETIQNLCHDLDRPVPELSLSLNDLPSNDFNYIFASLPEFYDRVKKRDNNYESLGFEHGSGGPCFVSAVPGSFYGRLFPRRSLHFVHSSSSLHWLSQVPCGEVNKKDGVVITADLDNRGKIYLSKTSPKSAHKVYALQFQTDFSVFLRSRSEELVPGGRMVLSFLGRSSPDPTTEESCYQWELLAQALMSLAKEGIIEEENIDAFNAPYYAASPEELKMAIEKEGSFSIDRLEISPVDWEGGSISDDSYDIVRFKPEALASGRRVAKTIRAVVEPMLEPTFGQKVMDELFERYAKLVGEYVYVSSPRYTIVIVSLLRMG

SEQ ID NO: 51 - protein, 392 aa, Brassica juncea
  misc_feature: Public GI no. 55442027
MEVMRILHMNKGNGETSYAKNSIVQSNIISLGRRVMDEALKKLMIRNSEILSFGIADLGCSSGPNSLLSISNIVETIQNLCHDLDRPVPELSLSLNDLPSNDFNYIFASLPEFYDRVKKRDNNYESLGFEHGSGGPCFVSAVPGSFYGRLFPRRSLHFVHSSSSLHWLSQVPCGEVNKKDGVVITADLDNRGKIYLSKTSPKSAHKVYALQFQTDFSVFLRSRSEELVPGGRMVLSFLGRSSPDPTTEESCYQWELLAQALMSLAKEGIIEEENIDAFNAPYYAASPEELKMAIEKEGSFSIDRLEISPVDWEGGSISDDSYDIVRFKPEALASGRRVAKTIRAVVEPMLEPTFGQKVMDELFERYAKLVGEYVYVSSPRYTIVIVSLLRMD

SEQ ID NO: 52 - protein, 389 aa, Capsicum annuum
  misc_feature: Public GI no. 77745528
MEVMRVLHMNKGNGETSYAKNSTAQSNIISLGRRVMDEALKKLMMSNSEISSIGIADLGCSSGPNSLLSISNIVDTIHNLCPDLDRPVPELRVSLNDLPSNDFNYICASLPEFYDRVNNNKEGLGFGRGGGESCFVSAVPSSFYGRLFPRRSLHSVHSSSSLHWLSQVPCREAEKEDRTITADLENMGKIYISKTSPKSAHKAYALQFQTDFLGFLRSRSEKLVPEGRMVLSFLGKRSLDPTTEESCYQWELLAQALMSMAKEGIIEEEKIDAFNAPYYAASSEELKMVIEKEGSFSIDRLEISPIDWEGGSISEESYDLVIRSKPEALASGRRVSNTIRAVVEPMLEPTFGENVMDELFERYAKIVGEYFYVSSPRYAIVILSLVRAG

SEQ ID NO: 53 - protein, 357 aa, Petunia x hybrida
  misc_feature: Public GI no. 28629495
MEVVEVLHMNGGNGDSSYANNSLVQQKVILMTKPITEQAMIDLYSSLFPETLCIADLGCSLGANTFLVVSQLVKIVEKERKKHGFKSPEFYFHFNDLPGNDFNTLFQSLGAFQEDLRKHIGESFGPCFFSGVPGSFYTRLFPSKSLHFVYSSYSLMWLSQVPNGIENNKGNIYMARTSPLSVIKAYYKQYEIDFSNFLKYRSEELMKGGKMVLTLLGRESEDPTSKECCYIWELLAMALNKLVEEGLIKEEKVDAFNIPQYTPSPAEVKYIVEKEGSFTINRLETSRVHWNASNNEKNGGYNVSRCMRAVAEPLLVSHFDKELMDLVFHKYEEIVSDCMSKENTEFINVIISLTKIN

SEQ ID NO: 54 - protein, 358 aa, Nicotiana tabacum
  misc_feature: Public GI no. 58201458
MKVVEVLHMNGGIGDISYAKNSLVQQKVILMTKPITEQAITDLYCSLFPQNLCIADLGCSSGANTFIVVSELIKIVEKERKKHGFQSPEFHFNFNDLPGNDFNTIFQSLDIFQQDLRKQIGEEFGPCFFSGVSGSFYTRLFPSNSLHFVHSSYSLMWLSQVPDAVENNKGNIYMASTSPPSVIKAYYKQYEKDFSNFLKYRSEELMKGGKMVLTFLGRESEDPTSKECCYIWELLAMALNELVVEGLIEEEKVDSFNIPQYTPSPADVKYVVEKEGSFTINQLEATRVHWNACNDKYKNVGYSVSRCMRAVAEPLLVSQFGEELMDLVFHKYEQIISECMSKAQTEFTNVIVSLTKTN

SEQ ID NO: 55 - protein, 364 aa, Antirrhinum majus
  misc_feature: Public GI no. 9789277
MKVMKKLLCMNIAGDGETSYANNSGLQKVMMSKSLHVLDETLKDIIGDHVGFPKCFKMMDMGCSSGPNALLVMSGIINTIEDLYTEKNINELPEFEVFLNDLPDNDFNNLFKLLSHENGNCFVYGLPGSFYGRLLPKKSLHFAYSSYSIHWLSQVPEGLEDNNRQNIYMATESPPEVYKAYAKQYERDFSTFLKLRGEEIVPGGRMVLTFNGRSVEDPSSKDDLAIFTLLAKTLVDMVAEGLVKMDDLYSFNIPIYSPCTREVEAAILSEGSFTLDRLEVFRVCWDASDYTDDDDQQDPSIFGKQRSGKFVADCVRAITEPMLASHFGSTIMDLLFGKYAKKIVEHLSVENSSYFSIVVSLSRR

SEQ ID NO: 56 - protein, 365 aa, Medicago truncatula
  misc_feature: Public GI no. 87241303
MATEGILHMKGGVGETSYENNSSLQRKVIMEVKTILEENMISIVSNKSIIKGCWKIADLGCSSGPNTLMAISNILNIINKTSLKLNNGISPVFQIYLNDLFENDFNTIFKLLPDFYQQKKGENVGECFICATPGNFYGRLFPNNYINFFHSSYSLHWLSQAPKDLTKNGEPLNKGNIYISRTSPPSVYEAYFKQFERDFKYFLKSRFEELTSDGVMALTFIGRETTITSAQGVIGMVLNEMVKEGLVEEEKLDLFDFPAYHPTVKEVSQLIEAEGSFTLQTIKTFKMGWDANLEKDNVDYVVDSKMRGEFIAKYHRAVYEPLLIAGFGENIMDELFSRFAKLIAQLIEIETLEFTNIVLFMTKNP

SEQ ID NO: 57 - protein, 359 aa, Clarkia breweri
  misc_feature: Public GI no. 6002712
MDVRQVLHMKGGAGENSYAMNSFIQRQVISITKPITEAAITALYSGDTVTTRLAIADLGCSSGPNALFAVTELIKTVEELRKKMGRENSPEYQIFLNDLPGNDFNAIFRSLPIENDVDGVCFINGVPGSFYGRLFPRNTLHFIHSSYSLMWLSQVPIGIESNKGNIYMANTCPQSVLNAYYKQFQEDHALFLRCRAQEVVPGGRMVLTILGRRSEDRASTECCLIWQLLAMALNQMVSEGLIEEEKMDKFNIPQYTPSPTEVEAEILKEGSFLIDHIEASEIYWSSCTKDGDGGGSVEEEGYNVARCMRAVAEPLLLDHFGEAIIEDVFHRYKLLIIERMSKEKTKFINVIVSLIRKSD

SEQ ID NO: 58 - protein, 365 aa, Camellia sinensis
  misc_feature: Public GI no. 59611829
MKEVKEALFMNKGEGESSYAQNSSFTQTVTSMTMPVLENAVETLFSKDFHLLQALNAVDLGCAAGPTTFTVISTIKRMVEKKCRELNCQTLELQVYLNDLPGNDFNTLFKGLQSKVVGNKCEEVSCYVVGVPGSFHGRLFPRNSLHLVHSCYSVHWLTQAPKGLTSKEGLALNKGKIYISKTSPPVVREAYLSQFHEDFTMFLNSRSQEVVPNGCMVLILRGRLSSDPSDMGSCSTWELLAVAIAELVSQGLIDEDKLDTFNVPSYFPSLEEVKDIVERNGSFTIDHMEGFELDSPEMQEDDKWVRGEKFATVARAFTEPIISNQFGHEIMDKLYEKFTHIVVSDLEAKIPKITSIILVLSKIVG

SEQ ID NO: 59 - DNA, 252 bp, Artificial Sequence
  Synthetic promoter sequence
cgcaggcccgatcggcctaaataattatagtcataagaggacccaaataaataaataatgggattctctgaggtgtatttttttatgcacacgtaaaagcgtgaaagatttgtaaacctactttatatatatacttccatctctttgtctttgttacaatttgaatcagagagaaattaagaagcgaaaaacaaagaacgagaggaggcgagaggtatagaagaatattccttgtggccggcaaggccaatc

SEQ ID NO: 60 - DNA, 389 bp, Artificial Sequence
  Synthetic promoter sequence
cgcaggcccgatcggcccaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatatatatacacgtaataagaaggtgaatgaatctcacagcttacttgttctaaggcttccaataacgaaaacagtaggccggcaaggccaatc

SEQ ID NO: 61 - DNA, 999 bp, Arabidopsis thaliana
  misc_feature: Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 62 - DNA, 999 bp, Arabidopsis thaliana
  misc_feature: Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 63 - DNA, 1000 bp, Arabidopsis thaliana
  misc_feature: Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 64 - DNA, 999 bp, Arabidopsis thaliana
  misc_feature: Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 65 - DNA, 1000 bp, Arabidopsis thaliana
  misc_feature: Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 66 - DNA, 3000 bp, Arabidopsis thaliana
  misc_feature: Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtcaatgtcaagcatacagctaaaatatcattatctaatattaagagtaaaacaagataattaaaaattgaaacaacaccatatttttatagctttacttatcgtatttttctagtcttcatggtaattgtgttgctttattttgtttataaatgaatttggttcgaccagatagtctaatatcagtttttaaacactggttttaataaaatcatatgtcggcaattcaacctgttacgttgtatgattgtatcctagtcaaataggggaggaggtactagtcgtttcaattagtttacgtaatcaatccaaagaaactataagctataaagatcctcaatttgttggttacaataaaaacaacagttgtcaaaatttatgtttataaaaagtaataactatgttccttcccatatagagcaaagtacctcaggataggcaaaccgtacttaatagcccttattcataatttgatccaactcttccccacaaaattgcaactgatgaagtcaatacttgtatagtgagtcaagctataaatgtctagtgatagttttgtctcttaaaaggttaacaaaagttatgacaagctgaaaaatcagagtttgctaggagtattacttacagttatcagtttaagtatcacatttatagtattgtatacaatgattcttaaattccaccttttccgtgcgaaaccaaattttctattggaaacatagaatgtaaacaaaaatatgggacgttgtccgttccaacattaaccaaacttgtctattactaatattcgtgttggtttgatgttggatgtctaaattcgttgaatcatgtgtctcttgacgaaatatgcatcttcttatttcttagtatagatgcactttatcattcttttagtacatgcttaatttttttttttaaaatatgttgattgtcatattgccaaaagtatgaattaaagacgcacatctaacacaagttagcagccgtaaatccttccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 67 - DNA, 999 bp, Arabidopsis thaliana
  misc_feature: Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 68 - DNA, 3156 bp, Populus balsamifera subsp. trichocarpa
  misc_feature: Ceres ANNOT ID no. 1494370
atggctcctaaaggtgacaatgttgttgtgtctagtatgaagcttgagaagttgctttgcatgaagggaggcaaaggagaggcaagctatgccaataactctcaagctcaggttactgatttctcattccattcttgcatttctttctcatggttcaagtttcctgacagttgtatatataaatgttttattgtggacatgtatatgcgtatcaacagctgattaggttgttaaattaattcctttaggagctagctaggttctatcagttaactatgaactctcttgatcaggagttcagtcttaccgaacctttttatgtagacgagttttaaactaactcctgagtatccatgcaggccttacatgctcgatccatgcttcaccttctggaagaaaccctagatagggtgcacctaaactccccagaattccctttccaggtggcggacttagggtgctcatctggcaacaacaccatccacatcattgatgtcatcatcaagcatatgatcaagcgattcgagtcatccggactcgagccgccggagttctctgcttttttcgccgatctccctagcaatgacttcaacacccttttccagctcctccctcccccggccaattatgggggtagtatggaagagtgcctagctgctagtggccatcgaaactattttgcagctggagttcccggctcctttcaccggaggctttttccggcaagatcaattgacgttttccactcggcattttccttgcactggctctctcaggttttttttccccccttcaaactttgttttgcatgtttagtcaagggatagcttttcttttttttaagttttcttatgaatggctaggtgaagcattaatgctacagaatatgcaaacatttttaaataaatattgaccaggaagtgccaataactcgttgctctgtgttacagaactctataaaaatagcttggtcatgtttttgaactagctagtggcaatacggtataggcaaaggaatatatagctaagtaaataagaataaaataattaatactctacataatcttttgtcggtttttagatggtcaaaagtaacgactccatggttattatcatcctaacaagtgttgaaagaaagtgtatgaaatcttgggtgtgtaaattcaaggcgtgtttgattttcaaagcatctttttggaggtgtgttgagtgggtggatgctgtgcccgatagagacaggaaatgaggagagaattctgggaccttgagatttgattaaattaaatatttattttgtgccatgaaattcatcaaagttttgaggtataattaactagctagctagatgagtctctgtattaatcaagatcgactcatcattaagttatgaaaacttgcttagattttaaagtgagcccttgaataatttaatttccgtgtgcaataatccatgaaaaaaacttggattagcgtagttaatcactactttttctcagaaaagttgattaattagcacctgacattagatcaaaagtcagttaagctaaattattaggggtatctaagagcatgcatcgatcgccagcaagcaaatcaaattgtaagttgagcaaattaattacaattaattactcaaagtgcatggcctgaattgatcacgaccaataatcttgcaggtgccggagtgtgttctagataagagatcagcagcatataacaaggggagggtgtttatccacaatgcaagcgagtccacaacaaatgcatacaagaaacagttccaaacagacctcgccggcttcctaagtgcaagatctcaagaaatgaagagcggtgggtccatgtttcttgtctgcttgggcagaacttctgccgaccccacagaccaaggtggggctggcctcctcttcgggacccactttcaggatgcctgggatgatcttgtccaggaggtaatcatcatttctcaagaaatgctcctaattcttatgtttgagtgtttgaacagtctttatccattttgccttcgagaccaaaaagcaagaaaacaccatgcagcaggggatatgattaaacgcagagcttacaagaccttattatgagatgtcactggtcattattagtcctattcaaattgaaccgttgatgcgataggttctagccatccatactctgatctccatgtcggcaatatgataactgtttgcagatgggcaggccatattattgatcgccatgctagatttatacaagtgctcacttttctgtagcacaacacaatcatagtatactttttttatctagacaagaaactcatttttggatccattatatcataaaatgatggattgtggggctcaaaccattgagtttgagtacatacgtatgatatcatgtctcttaatcaactactctaatcagaatggtacgtactatagattataataattaggttttcatggctttgaagcctatgaatgaatgaatatctgtgtagcttgtgccgaatacagaggcttgcttactggtattttctatgctaataattggacctttgattaccattatctaagttttaagtactgtgtgggatagctagctgtcggcccatcaggcagctgctgttaatcggcctggtttgcagttgaatctaagtgggccgagagtgaaaaggctcgttccctaggttttttttttattattatatttttggagaaaacctagctagctttagttatgtgcacttctcaaaagggtttcgggacactaactgatgaatggtatgctgctaattaatctcagggtctgattactagcgagaagcgtgacaatttcaacattccagtgtatgcaccaagcctccaagacttcaaggaagtagtggaagccaatggctcatttaccatagacaagctcgaggttttcaaaggagggagccctctggtggttaaccatcctgacaacgaagctgaggtcagccgggccatggctaattcctgccggagtgtggccggggtccttgttgatgcccacattggtgatgggctaagcgaggagttgttcttacgggtggagcaccgagccaaaagccatgcgaaagagcttctagagaagctgcaattcttccatatagttgcatctctttcttttgcttga